(19) United States
(12) Patent Application Publication
(10) Pub. No.: US 2023/0410950 A1
(43) Pub. Date: Dec. 21, 2023

(54) CHEMICAL REACTION GRAPH COMPRESSION SOFTWARE, CORRESPONDING METHOD AND ASSOCIATED DATA APPLICATIONS

(71) Applicant: Firmenich SA, Satigny (CH)

(72) Inventors: Guillaume GODIN, Satigny (CH); Ruud VAN DEURSEN, Satigny (CH)

(21) Appl. No.: 18/247,717

(22) PCT Filed: Oct. 26, 2021
(86) PCT No.: PCT/EP2021/079732

§ 371 Date: Apr. 3, 2023

(30) Foreign Application Priority Data
Oct. 26, 2020 (EP) 20203945.9
Apr. 30, 2021 (EP) 21171478.7

(51) Int. Cl. G16C 20/10; G16C 20/70; G16C 20/80; G06N 20/00

(52) U.S. Cl. CPC G16C 20/10; G16C 20/70; G16C 20/80; G06N 20/00

(57) ABSTRACT

The chemical reaction encoding method (100) for one-step, multi-step and equilibrium reactions, comprises:—a step (105) of receiving, upon a computer interface, a chemical reaction graph comprising at least one chemical reaction reagent and at least one chemical reaction product, —a first step (110) of encoding, by a computing device, said chemical reaction graph describing the structure of at least one said reagent and said product, —a step (115) of determination, by a computing device, of changing bonds within the encoding representative of the chemical structures of at least one said reagent and said product, —a second step (120) of encoding, by a computing device, in a single string of characters, for at least one changing bond determined, at least one character representative of an atom subject to the change of bond, at least one character representative of the type of changing bond determined and at least one character representative of an atom resulting from the change of bond, in which a changing bond is encoded by a set of two characters representative of the changing bond determined, the first character being representative of the reagent bond and the second character being representative of the product bond, each character being selected in a library of bijective characters wherein one character is representative of one changing bond type and—a step (125) of providing, upon a computer interface, the string of characters corresponding to the encoding of changing bonds of the chemical reaction.

### TECHNICAL FIELD OF THE INVENTION

The present invention relates to a chemical reaction graph compression software, the corresponding method, to a chemical reaction graph format, to a chemical reaction dataset augmentation method, to a chemical reaction dataset preprocessing method, to a training method for a classifier, transformer or regressor, to a chemical reaction bond evolution prediction method, to a chemical reaction generation method, to a computer-implemented classifier, transformer or regressor and to a related computer program. It applies, in particular, to the fields of organic chemistry, including, but not limited to pharmaceutics, perfumery, flavours, cleaning products, fragrance design and olfactometry, perfumery, fine fragrance perfumery and flavour design.

### BACKGROUND OF THE INVENTION

In the field of chemical species and chemical reaction digital modelling, one of the key encoding systems is line notation, such as the simplified molecular-input line-entry system (SMILES) format. Such a format is abundantly documented, including on mainstream sources such as the collaborative encyclopedia Wikipedia.

While such formats, such as SMARTS and SMIRKS, have been instrumental in the understanding and capacity to model chemical interactions, drawbacks are starting to appear:
the excessive number of characters, and the corresponding physical memory space occupied, needed to encode a chemical reaction implies longer transmission and processing times for systems using such formats,
underperformance of such a format in machine learning applications due to excessive amount of possible irrelevant information stored within the format,
in older formats such as SMARTS or SMIRKS string, which are composed of dot-separated reagents, dot-separated agents (enablers of the reaction, conditions) and dot-separated products, require explicit atom mapping to define the reaction. In our new short CRS format, needing for large amount of information,
no simple and compact encoding of reversibility,
no simple and compact encoding multi-step reactions, i.e. A>B>C,
no simple and compact encoding of equilibrium reactions, i.e. A< >B or A>B>A,
no simple and compact encoding of reaction mechanisms, i.e. A>T>B, where T defines the transition state of the reaction,
do not allow for reaction classification and data cleaning, thus reducing the signal-to-noise ratio when the data is used,
are ambiguous in terms of characters used, which reduces the signal-to-noise ratio when the data is used,
no simple and compact encoding of biochemical pathways, composed of multiple intermediates,
no simple and compact encoding of stereochemistry and
no capacity to display changes of stereoisomerism on a tetravalent chiral centre.

Furthermore, modern chemical reaction research and development cycles require more advanced tools than the typical trial and error approach or other approaches based solely on already existing knowledge within an organisation. In such a context, machine learning appears to be a cornerstone to the optimisation of this research and development cycle. However, the performance of machine learning models is limited by the quality of the input data. As of today, there is no satisfying way to produce machine learning models to predict chemical reaction behaviour or to generate new chemical reactions in an autonomous manner.

### SUMMARY OF THE INVENTION

The present invention is intended to remedy all or part of these disadvantages.

To this effect, according to a first aspect, the present invention aims at a chemical reaction graph compression software for one-step, multi-step and equilibrium reactions, executing instructions corresponding to the following steps:
a step of receiving, upon a computer interface, a chemical reaction graph comprising at least one chemical reaction reagent and at least one chemical reaction product,
a first step of encoding, by a computing device, said chemical reaction graph describing the structure of at least one said reagent and said product,
a step of determination, by a computing device, of changing bonds within the encoding representative of the chemical structures of said at least one reaction reagent and said product,
a second step of encoding, by a computing device, in a single string of characters, for at least one changing bond determined, at least one character representative of an atom subject to the change of bond, at least one character representative of the type of changing bond determined and at least one character representative of an atom resulting from the change of bond, in which a changing bond is encoded by a set of two characters representative of the changing bond determined, the first character being representative of the reagent bond and the second character being representative of the product bond, each character being selected in a library of bijective characters wherein one character is representative of one bond type and
a step of providing, upon a computer interface, the string of characters corresponding to the encoding of changing bonds of the chemical reaction.

Such provisions focus on the locations of the reagents where a change of bond happens, thus allowing for highly performative encoding by the limitation of material to encode. The resulting code, more compact, limits the physical memory usage. Furthermore, focusing on change of bond allows for machine learning applications to target only the relevant parts of the chemical reaction, thus allowing for increased speed and accuracy.

Additionally, this formatting allows for the modulization of multistep reactions or chemical equilibrium reactions, i.e. A< >B as a pseudo-two-step reaction of by writing of the individual reactions A>B and B>A or as a multistep reaction A>B>A.

Furthermore, the resulting formatting is reversible, allows the definition of equilibrium reactions, allows for the encoding of reaction mechanisms, is unambiguous, allows for reaction classifications and data cleaning, allows encoding of stereochemistry changes and can indicate changes to tetravalent chiral centres.

In particular embodiments, the second step of encoding is configured to embed the two characters' representative of the changing bonds determined in between two neutral tag characters representative of the presence of an encoding of said changing bonds.

In particular embodiments, multistep reactions, represented by a succession of change of bonds between two atoms, are encoded by a succession of single characters, each single character being representative of the successive state of a bond between said two atoms, the order of the characters being representative of the order of changes of bonds between said two atoms.

Such embodiments allow for the automatic recognition, by an element of software, that the two characters representative of the changing bonds are to be isolated as being non-representative of the atoms as such.

According to a second aspect, the present invention aims at a chemical reaction graph compression method for one-step, multi-step and equilibrium reactions, comprising:
a step of receiving, upon a computer interface, a chemical reaction graph comprising at least one chemical reaction reagent and at least one chemical reaction product,
a first step of encoding, by a computing device, said chemical reaction graph describing the structure of at least one said reagent and said product,
a step of determination, by a computing device, of changing bonds within the encoding representative of the chemical structures of at least one said reaction reagent and said product,
a second step of encoding, by a computing device, in a single string of characters, for at least one changing bond determined, at least one character representative of an atom subject to the change of bond, at least one character representative of the type of changing bond determined and at least one character representative of an atom resulting from the change of bond, in which a changing bond is encoded by a set of two characters representative of the changing bond determined, the first character being representative of the reagent bond and the second character being representative of the product bond, each character being selected in a library of bijective characters wherein one character is representative of one changing bond type and
a step of providing, upon a computer interface, the string of characters corresponding to the encoding of changing bonds of the chemical reaction.

The benefits and advantages of this method correspond to the benefits of the software object of the first aspect of the present invention.

In particular embodiments, multistep reactions, represented by a succession of change of bonds between two atoms, are encoded by a succession of single characters, each single character being representative of the successive state of a bond between said two atoms, the order of the characters being representative of the order of changes of bonds between said two atoms.

In particular embodiments, the first step of encoding is configured to encode the chemical reaction graph into a line notation, the method further comprising, prior to the second step of encoding, a step of augmenting the line notation encoding.

Such embodiments allow for the increase in sample size, starting from a single chemical reaction graph. This is particularly useful in machine learning applications.

In particular embodiments, the second step of encoding comprises a step of extracting, by a computing device, of a bond table for reagents and products from a computer memory, said encoding being performed as a function of said bond table.

In particular embodiments, the second step of encoding comprises a step of removing, from the first encoding resulting from the first step of encoding, of at least one atom identifier from at least one reagent and/or product, each said atom being removed as a result of the step of determination in the event said atom and the associated bonds are located in a product and/or reagent that remains unchanged from reagent the reaction stage to the product stage of the chemical reaction.

Such embodiments allow for the greater compression of a chemical reaction format by limiting the notation of the reaction to the reaction site.

In particular embodiments, the method object of the present invention comprises a step of obtaining the products of the encoded chemical reaction by performing said chemical reaction in a physical device.

According to a third aspect, the present invention aims at an encoded chemical reaction comprising a string of characters that it is obtained by the method object of the second aspect of the present invention.

The benefits and advantages of this formatted chemical reaction graph correspond to the benefits of the method object of the second aspect of the present invention.

According to a fourth aspect, the present invention aims at a chemical reaction dataset augmentation method, comprising:
a step of receiving, upon a computer interface, a string of characters according to the encoding object of the third aspect of the present invention,
a step of reordering, by a computing system, the string of characters in order to shift at least one character representative of an atom and at least one string of at least one character representative of a change of bond associated to the corresponding atom and
a step of outputting, upon a computer interface, an augmented string of characters corresponding to the reaction initially encoded by the received string of characters.

Such provisions allow for the increase in sample size, starting from a single chemical reaction graph. This is particularly useful in machine learning applications.

In particular embodiments, the method object of the present invention comprises a step of associating, by a computing system, at least two strings of characters according to the format object of the third aspect of the present invention, each said string of characters being representative of the same chemical reaction graph.

Such provisions allow for the creation of multidimensional inputs that are particularly useful in machine learning applications.

According to a fifth aspect, the present invention aims at a chemical reaction dataset preprocessing method, comprising:
a step of receiving, upon a computer interface, a dataset of at least two chemical reaction graphs comprising at least one chemical reaction reagent and at least one chemical reaction product,
a step of compression of at least two chemical reaction graphs according to the method object of the second aspect of the present invention,
a step of determining, by a computing system, a distribution of chemical reaction classes within the encoded dataset,
a step of augmenting the dataset, according to the method object of the fourth aspect of the present invention, for at least one chemical reaction class as a function of the determined distribution and
a step of outputting, upon a computer interface, the preprocessed dataset.

Such provisions allow for the dynamic and smart augmentation of a dataset to optimise machine learning applications.

According to a sixth aspect, the present invention aims at a training method for a classifier, transformer or regressor, comprising:
inputting, upon a computer interface, a dataset of chemical reaction graphs encoded in the compressed encoding object of the third aspect of the present invention,
operating, by a computing system, a recursive neural network architecture configured to use, as input, the dataset of chemical reaction graphs to classify the chemical reaction bond evolution as a function of the input and
outputting, upon a computer interface, a trained classifier, transformer or regressor.

Such provisions allow for the optimal creation of a trained classifier, transformer or regressor as the chemical graph reaction format used significantly improves the quality of the generated models.

According to a seventh aspect, the present invention aims at a chemical reaction bond evolution prediction method, operating a classifier, transformer or regressor obtained by the method object of the sixth aspect of the present invention.

Such provisions allow for the prediction of the bond evolution of any input chemical reagents with accuracy.

According to an eighth aspect, the present invention aims at a chemical reaction generation method, operating a classifier, transformer or regressor obtained by the method object of the sixth aspect of the present invention.

Such provisions allow for autonomous generation of chemical reactions, with corresponding graphs and/or linear notation.

According to a ninth aspect, the present invention aims at a computer-implemented classifier, transformer or regressor, wherein the classifier, transformer or regressor is obtained by the method object of the sixth aspect of the present invention.

The benefits and advantages of this computer-implemented classifier, transformer or regressor correspond to the benefits of the method object of the sixth aspect of the present invention.

According to a tenth aspect, the present invention aims at a computer program, comprising instructions to operate a method object of either one of the sixth, seventh or eighth aspects of the present invention.

The benefits and advantages of this computer program correspond to the benefits of the method object of the corresponding sixth, seventh or eighth aspect of the present invention.

### BRIEF DESCRIPTION OF THE DRAWINGS

Other advantages, purposes and particular characteristics of the invention shall be apparent from the following non-exhaustive description of at least one particular embodiment of the present invention, in relation to the drawings annexed hereto, in which:

FIG. 1 represents, schematically, a first particular succession of steps representative of the method object of the present invention,

FIG. 2 represents, schematically, a chemical reaction graph encoded by the method object of the present invention,

FIG. 3 represents, schematically, a second particular succession of steps representative of the method object of the present invention,

FIG. 4 represents, schematically, a third particular succession of steps representative of the method object of the present invention,

FIG. 5 represents, schematically, a fourth particular succession of steps representative of the method object of the present invention,

FIG. 6 represents, schematically, the states of encoding of a chemical reaction via the software object of the present invention,

FIG. 7 represents, schematically, the states of encoding of an equilibrium chemical reaction via the software object of the present invention,

FIG. 8 represents, schematically, instructions of a particular set of instructions of the software object of the present invention,

FIG. 9 represents, schematically, the states of encoding of a multistep chemical reaction via the software object of the present invention,

FIG. 10 represents, schematically, the states of encoding of an equilibrium chemical reaction via the software object of the present invention,

FIG. 11 represents, schematically, a particular succession of step relative to the method of augmenting object of the present invention,

FIG. 12 represents, schematically, a particular succession of step relative to the method of generating chemical reaction graphs object of the present invention,

FIG. 13 represents, schematically, a first particular succession of step relative to the method of training a classifier object of the present invention,

FIG. 14 represents, schematically, a second particular succession of step relative to the method of training a classifier object of the present invention and

FIGS. 15 to 27 represent, schematically, a particular example and associated results of the generation method object of the present invention.

### DETAILED DESCRIPTION OF THE INVENTION

This description is not exhaustive, as each feature of one embodiment may be combined with any other feature of any other embodiment in an advantageous manner.

Also, various inventive concepts may be embodied as one or more methods, of which an example has been provided. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

The indefinite articles ‘a’ and ‘an’, as used herein in the specification and in the claims, unless clearly indicated to the contrary, should be understood to mean ‘at least one’.

The phrase ‘and/or’, as used herein in the specification and in the claims, should be understood to mean ‘either or both’ of the elements so conjoined, i.e. elements that are conjunctively present in some cases and disjunctively present in other cases. Multiple elements listed with ‘and/or’ should be construed in the same fashion, i.e. ‘one or more’ of the elements so conjoined. Other elements may optionally be present other than the elements specifically identified by the ‘and/or’ clause whether related or unrelated to those elements specifically identified. Thus, as a non-limiting example, a reference to ‘A and/or B’, when used in conjunction with open-ended language such as ‘comprising’ can refer, in one embodiment, to A only (optionally including elements other than B); in another embodiment, to B only (optionally including elements other than A); in yet another embodiment, to both A and B (optionally including other elements); etc.

As used herein in the specification and in the claims, ‘or’ should be understood to have the same meaning as ‘and/or’ as defined above. For example, when separating items in a list, ‘or’ or ‘and/or’ shall be interpreted as being inclusive, i.e. the inclusion of at least one, but also including more than one, of a number or list of elements, and, optionally, additional unlisted items. Only terms clearly indicated to the contrary, such as ‘only one of’ or ‘exactly one of’, or, when used in the claims, ‘consisting of’, will refer to the inclusion of exactly one element of a number or list of elements. In general, the term ‘or’ as used herein shall only be interpreted as indicating exclusive alternatives (i.e. ‘one or the other but not both’) when preceded by terms of exclusivity, such as ‘either,’ ‘one of,’ ‘only one of’, or ‘exactly one of’. ‘Consisting essentially of,’ when used in the claims, shall have its ordinary meaning as used in the field of patent law.

As used herein in the specification and in the claims, the phrase ‘at least one’, in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase ‘at least one’ refers, whether related or unrelated to those elements specifically identified. Thus, as a non-limiting example, ‘at least one of A and B’ (or, equivalently, ‘at least one of A or B’, or, equivalently ‘at least one of A and/or B’) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

In the claims, as well as in the specification above, all transitional phrases such as ‘comprising,’ ‘including,’ ‘carrying,’ ‘having,’ ‘containing,’ ‘involving,’ ‘holding,’ ‘composed of’, and the like are to be understood to be open-ended, i.e. to mean including but not limited to. Only the transitional phrases ‘consisting of’ and ‘consisting essentially of’ shall be closed or semi-closed transitional phrases, respectively, as set forth in the United States Patent Office Manual of Patent Examining Procedures, Section 2111.03.

It should be noted at this point that the figures are not to scale.

It should be noted that the terms ‘computer interface’ are to be understood as any type of human-machine interface, such as a Graphic User Interface (GUI) associated to an input means, such as a keyboard, mouse or a touchscreen, for example. These terms also refer to any software or digital interface, such as an application programming interface (‘API’) for example, or any other type of digital input/output means or software.

It should be noted that the terms ‘computing device’ or ‘computing system’ are to be understood as any electronic computation means, such as a microprocessor preferably associated to a computer memory and the required input/output subsystems. The particular architecture of the computing system used in the description below is unimportant considering the present invention. That is to say, such a computing system may be distributed, integrated, using a client-server architecture or using local and/or distant computing resources. Data stored and accessed may be stored in traditional databases, in computer memories or in distributed databases.

It should be noted that the terms ‘chemical reaction graph’ designate a modelling of a chemical reaction in graph format in which each molecule (reagent and product) is modelled into a graph whose vertices correspond to the atoms of the compound and edges correspond to chemical bonds. Chemical reaction graphs thus model the structural formula of a chemical compound in terms of graph theory. Typically, a molecular graph comprises atom digital identifiers and bond digital identifiers allowing for the graph to be built. These digital identifiers may be graphically translated into labels and vertices. Such digital identifiers may be stored in a digital storage device, such as a computer memory, a server database or a distributed database.

It should be understood that the term ‘character’ refers to any symbol (whether alphabetical or not) that can be used to generate a code from an input. Typically, a character can be an ASCII (for ‘American Standard Code for Information Interchange’) code representative of a character. This is, however, not limitative with respects to the present invention.

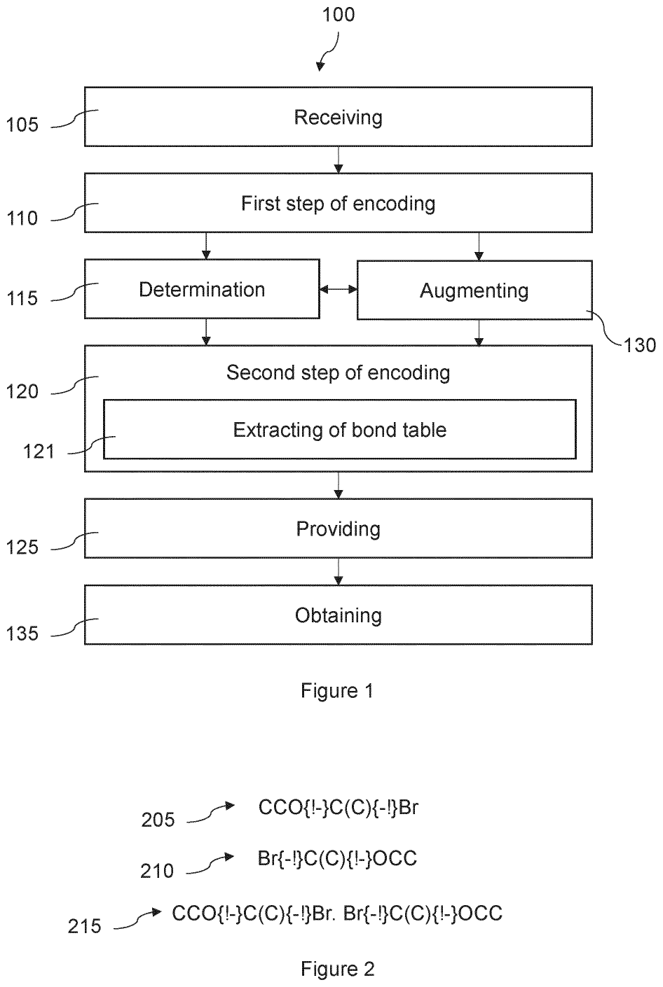

FIG. 1 shows, for example, a succession of steps corresponding to instructions of a chemical reaction graph compression software for one-step, multi-step and equilibrium reactions, this software executing instructions corresponding to the following steps:
a step 105 of receiving, upon a computer interface, a chemical reaction graph comprising at least one chemical reaction reagent and at least one chemical reaction product,
a first step 110 of encoding, by a computing device, said chemical reaction graph describing the structure of at least one said reagent and said product,
a step 115 of determination, by a computing device, of changing bonds within the encoding representative of the chemical structures of said at least one reaction reagent and said product,
a second step 120 of encoding, by a computing device, in a single string of characters, for at least one changing bond determined, at least one character representative of an atom subject to the change of bond, at least one character representative of the type of changing bond determined and at least one character representative of an atom resulting from the change of bond, in which a changing bond is encoded by a set of two characters representative of the changing bond determined, the first character being representative of the reagent bond and the second character being representative of the product bond, each character being selected in a library of bijective characters wherein one character is representative of one bond type and
a step 125 of providing, upon a computer interface, the string of characters corresponding to the encoding of changing bonds of the chemical reaction.

The step 105 of receiving is performed, for example, using any type of computer interface. During this step 105 of receiving, a digital resource is received, said digital resource being representative of a chemical reaction graph. A ‘digital resource’ is to be understood in the broadest way possible, that is a structured set of data. Such a digital resource can be a file stored within a computer memory or generated when required. Alternatively, a digital address for a file can be received instead of the file as such.

Alternatively, during the step 105 of receiving, digital identifiers corresponding to at least one reagent and at least one product are received. Such a digital identifier can be either a digital resource representative of a reagent or product or any pointer to said digital resource. Such a digital identifier may be an address in a database, for example, or a natural language string representative of said reagent or product. In other variants, the digital identifier is a component of a GUI that is actionable by a user and which, once activated, triggers the input of an associated resource and/or the address of said resources.

The step 105 of receiving may be triggered by the user or automatic input.

The first step 110 of encoding is performed, for example, by a computing system configured to run a dedicated software. This step 110 of encoding may be performed, for example, similarly to the way the SMILES format of a chemical reaction graph is generated. During this step 110 of encoding, the chemical reaction graph is preferably encoded into a string of characters in the ASCII format.

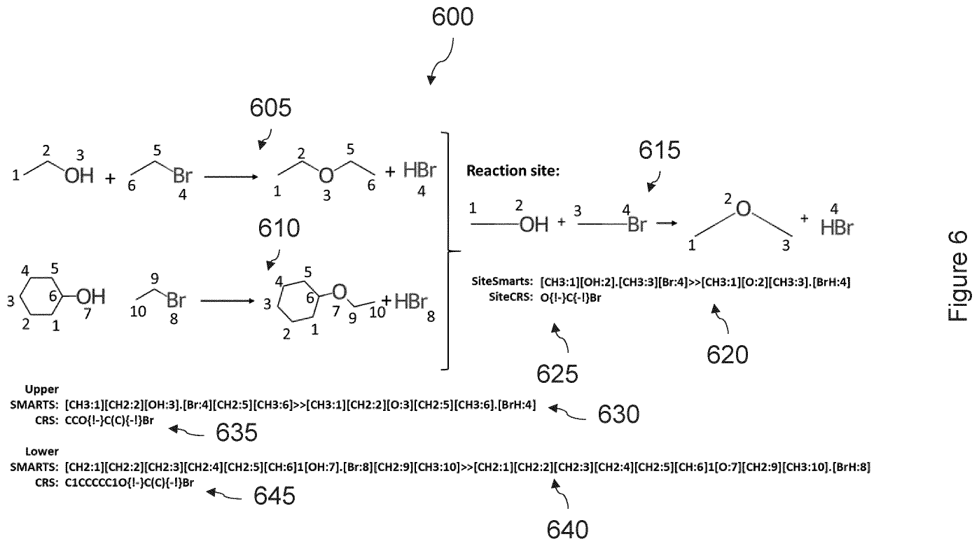

Alternatively, the first step 110 of encoding is configured to provide a line notation using the SMARTS (for ‘SMILES arbitrary target specification’) variant of the SMILES encoding format. The SMARTS encoding format is a language for specifying substructural patterns in molecules. FIG. 6 shows a result of such a first step 110 of encoding in regard to references 630 and 640 for reactions 605 and 610 respectively.

The step 115 of determination is performed, for example, by a computing system configured to run a dedicated software. During this step 115 of determination, several options may be implemented:
either requiring human input, upon a computer interface, in mapping the atoms and bonds in the chemical reaction graph or
automatically mapping the atoms and bonds, by a computing system, in the chemical reaction graph and in either case then
comparing, by a computing system, the product molecular chemical graphs to the reagent molecular chemical graphs in order to detect changes in molecular structures, either due to atom changes for a specific mapped location in the molecular graph or bond changes relative to said mapped atom or any other atom in the associated molecule and
classifying, by a computing system, the identified change of bond among a list of preset types as a function of the result of the step of comparison.

Such embodiments, using superposition comparison are typically used in contemporary solutions. However, these approaches typically lack in certainty of the mapping obtained as they look for structural minimum commonalities which, for example, if an oxygen molecule is used as a reagent and produced as a product, will not detect this destruction-creation process.

More advanced embodiments make use of transformer machine learning algorithms.

Such a model can be trained by the data included USPTO-50 sets (or part thereof) from an article by Schneider et al. (Schneider, N.; Stiefl, N.; Landrum, G. A., What's What: The—Nearly—Definitive Guide to Reaction Role Assignment. J Chem Inf Model 2016, 56, 2336-2346) as well as for some calculations can also be used the training set data from Jaworksi et al. (Jaworski, W., Szymkuć, S., Mikulak-Klucznik, B. et al. Automatic mapping of atoms across both simple and complex chemical reactions. Nat Commun 10, 1434-2019).

Such a model can be tested against a test set that can be a part of the USPTO-sets not used for training as well as manually curated reactions. Additionally, a test set of 857 reactions from Jaworksi et al. can be used to test performance of the developed methods.

Such data may be curated before input. Furthermore, the data may be compressed and encoded according to the method 100 object of the present invention prior to use as training/test data.

The transformer architecture as described in any of the following publications study can for example be used:
Vaswani, A., et al. Attention Is All You Need. Preprint at https://arxiv.org/abs/1706.03762 (2017),
Schwaller, P., et al. Molecular transformer: a model for uncertainty-calibrated chemical reaction prediction. ACS Cent. Sci. 5, 1572-1583 (2019) and/or
Tetko, I. V., Karpov, P., Van Deursen, R. et al. State-of-the-art augmented NLP transformer models for direct and single-step retrosynthesis. Nat Common 11, 5575 (2020).

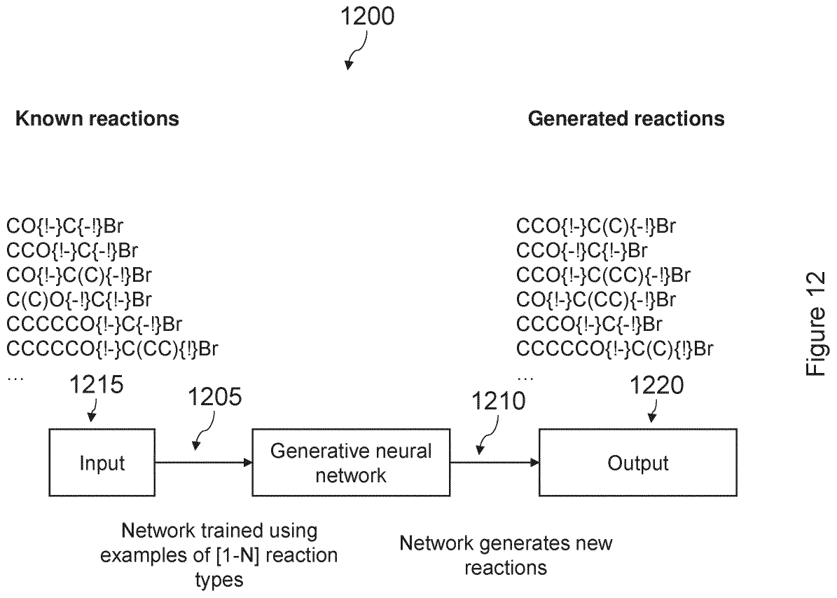

Namely the transformer consists of six layers and eight heads (6×8). The training of the model is restricted to 100 epochs and used a batch size of 3000 characters. The input data were reaction data (both reagents and products) in SMIRKS format while the targets were the respective chemical reaction graphs compressed and encoded according to the method 100 object of the present invention. Both input and target sequences can be augmented, such as shown in FIG. 12, which increases the data diversity and eliminates the effect of neural network overfitting. The data for model training and test can be augmented 5× and 20× times, respectively, for example.

The transformer model can generate multiple predictions for a given input data using a beam search. Using a beam search to n=10 and thus receiving ten predicted compressed and encoded chemical reaction graphs according to the method 100 object of the present invention (CRS) for each input reaction. Since a used 20× data augmentation can be used for each reaction a total number of up to 200 predicted CRSs can be calculated for each analysed reaction.

Further post-processing may occur, such as
filtering some calculated CRSs before further analysis due to obvious format errors,
mass balancing of reactants and/or products, to check that all reactants and reagents produced by decomposing CRS are present in the initial reaction.

Such a transformer model may provide results such as:

| | | | | | | | USPTO-50-train + | | | USPTO-50-train + | | |
|---|---|---|---|---|---|---|---|---|---|---|---|---|
| | USPTO-50-train | | | USPTO-50-train + | | | natcom train + | | | natcom train + all | | |
| Model | (43 k reactions) | | | nature train | | | signatures | | | signatures | | |
| | setb_ | | | setb_nature_ | | | setb_nature_sign_t | | | setb_sign_nature_ | | |
| intern. | a | | | train_a | | | rain_train_a | | | all_train_a | | |
| Test set | Cov | Prec | Tot | Cov | Prec | Tot | Cov | Prec | Tot | Cov | Prec | Tot |
| SetB | 99.9 | 99.9 | 99.8 | 99.9 | 100 | 99.9 | 99.9 | 100 | 99.9 | 99.9 | 100 | 99.9 |
| SetA | 96.3 | 99.7 | 96 | 97.2 | 99.7 | 96.9 | 97.5 | 98.8 | 96.3 | 98.8 | 99.7 | 98.5 |
| Nature | 67.3 | 98.1 | 66 | 73.4 | 97.4 | 71.5 | 79.7 | 97.3 | 77.6 | 95.4 | 98.8 | 94.2 |
| Test | | | | | | | | | | | | |

Implementing such transformer demonstrated an excellent performance when trained with such data. The model developed with 43.8k USPTO-50k training set demonstrated 99.9% Coverage and 100% Precision for the test set of 4,885 reactions. Thus, the transformer was able to correctly predict the atom mapping of all reactions from its test set. The performance of this model was lower for the manually annotated set A, for which it reached 96.7% coverage as well as Precision accuracy of 96.9%.

For the NatureTest set the Coverage was much lower, only 67.3%. The lower Coverage indicated that the Nature set contained reaction types, which were not present in the patents and/or were more complicated and the model was unable to produce one or more valid CRS for them. However, the same very high Precision accuracy was calculated. Thus, the Transformer model was able to exactly reproduce correct mapping if the produced CRS contained all components of the initial reaction data.

The increase of the diversity of the data by adding the NatureTrain set (n=548) improved the Coverage for NatureTest by about 7.3% as well as by above 1% for SetA. Additional boost of the Coverage for NatureTest was achieved when we added the simulated data generated using NatureTrain set. These data included 10 generated reactions per each initial reaction. This generation created a better representation of the rare reactions and increased the accuracy of models. However, even after the addition of simulated reactions the Coverage was below 80% for the NatureTest indicating that some reactions from this set were underrepresented both in USPTO patents as well as in the NatureTrain set.

To address this problem, it is possible to include the simulated reactions for the NatureTest, which boosts the coverage for this set up to 95% without decreasing the precision rate. The latter extension of the dataset also provided the best overall results for the Set A, for which Coverage increased to 98.9% and the precision rate achieved 97.4%. For the Set B the results did not change, and all three accuracy measures were about 100%.

The second step 120 of encoding is performed, for example, by a computing system configured to run a dedicated software. During this second step 120 of encoding, at least one of the changing bonds determined is encoded into a set of ASCII characters representative of the type of changing bond determined.

This second step 120 of encoding may comprise, for example, the following steps:
a step of parsing the encoded reaction graph resulting from the first step 110 of encoding,
a step of extraction of a bond table for reagents and products,
a step of generating the second encoding by assembling a reaction graph of the reagents and products and
optionally, the generated second encoding can be exported to a canonical linear notation string and write the bond with specified symbols, such as shown below.

The second step 120 of encoding said changing bonds in a single character string may be performed by associating each changing bond with a symbol, such as a succession of at least one character, describing the type of change of bond. This symbol is preferably associated with the neighbouring atoms in between which the change of bond is happening.

Such a symbol may be a four-character sequence composed of single bond characters for the reagent and product bond types, surrounded by curly brackets or other characters defined as neutral. A single to double bond change in a reaction is for instance written using the character sequence ‘{-=}’. The relationship linking character and represented change of bond is preferably bijective. The term ‘bijective’ refers here to the one-to-one relationship linking character and represented change of bond. It is understood that the term ‘character’ is to be understood as any symbol in a dictionary of symbols and not restrictively limited to alphanumeric characters. This means that a library of characters may be set up prior to the steps of encoding, in which each character represents a type of change of bond. Constituting this library can be performed manually or automatically. In particular embodiments, an algorithm can be trained to learn its own symbols. During the following step of encoding, the appropriate character or symbol is selected from the library as a function of the determined change of bond.

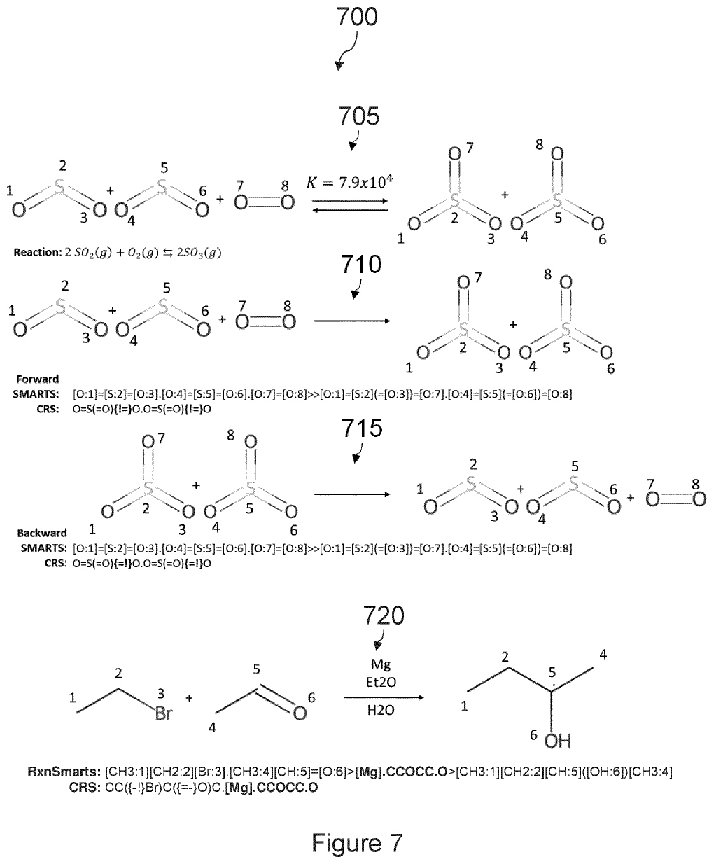

Apart from a format with both reagents and products in a single character string, the format stands out by the very short format that has no need for an explicit atom number to mark the reaction site. Indeed, the reaction is implicitly defined by the changing bonds. In a SMARTS chemical reaction, every reagent and product is defined by a new SMILES string. The order of the atoms may vary widely, including the canonical form. Consequently, one has to define explicit indices in the SMILES string to define which atoms are identical in reagents and products, e.g. [CH3:1][CH2:2][CH3:3], where: 1, :2 and:3 define the atom indices. Agents are typically not incorporated because they do not contribute to the net chemical modification. Agents and conditions may also vary between reactions and can be selected by users based on the type of reaction, such agents and conditions can be adjusted at the user's discretion such as exemplified in FIG. 7. An additional and important advantage of the proposed is the compressibility for large datasets. Indeed, this format is the shortest format known to describe a reaction. While this application focuses on reactions with bond breaking, creation and bond order modification, other types of bond changes may be encoded in this manner. Reaction with the formation and breaking of ionic bonds as well as purification, e.g. chiral separation, are not considered here. The latter group of reactions do not change the graph connectivity of the atoms. Such a separation can be written as: A.B>B as an example for a purification yielding B.

The corresponding table is representative of possible symbol selection for different types of changes of bonds:

| Symbol (character | | |
|---|---|---|
| sequence) | Reagent bond | Product bond |
| {!-} | None | Single |
| {!=} | None | Double |
| {!#} | None | Triple |
| {!:} | None | Aromatic |
| {-!} | Single | None |
| {-=} | Single | Double |
| {-#} | Single | Triple |
| {-:} | Single | Aromatic |
| {=!} | Double | None |
| {=-} | Double | Single |
| {=#} | Double | Triple |
| {=:} | Double | Aromatic |
| {#!} | Triple | None |
| {#-} | Triple | Single |
| {#=} | Triple | Double |
| {#:} | Triple | Aromatic |
| {:!} | Aromatic | None |
| {:-} | Aromatic | Double |
| {:=} | Aromatic | Triple |
| {:#} | Aromatic | Aromatic |

Bonds indicated with ‘None’ in reagent are product bonds formed during the reaction. Bonds indicated with ‘None’ in product are reagent bonds broken during the reaction.

In particular embodiments, the second step 120 of encoding is configured to encode a changing bond in a set of two characters representative of the changing bonds determined, the first character being representative of the reagent bond and the second character being representative of the product bond.

In particular embodiments, the second 120 step of encoding is configured to embed the two characters representative of the changing bonds determined in between two neutral tag characters representative of the presence of an encoding of said changing bonds.

An example of such output 205 is shown in FIG. 2.

The step 125 of providing is performed, for example, upon a GUI or via the use of an API.

FIG. 1 shows, furthermore, the method 100 implemented by the software disclosed above. This chemical reaction graph compression method 100 for one-step, multi-step and equilibrium reactions, comprises:
a step 105 of receiving, upon a computer interface, a chemical reaction graph comprising at least one chemical reaction reagent and at least one chemical reaction product,
a first step 110 of encoding, by a computing device, said chemical reaction graph describing the structure of at least one said reagent and/or said product,
a step 115 of determination, by a computing device, of changing bonds within the encoding representative of the chemical structures of at least one reaction reagent,
a second step 120 of encoding, by a computing device, in a single string of characters, for at least one changing bond determined, said changing bond into a string of at least one character associated to at least one character representative of an atom subject to the change of bond and at least one character representative of an atom resulting from the change of bond and
a step 125 of providing, upon a computer interface, the string of characters corresponding to the encoding of changing bonds of the chemical reaction.

In particular embodiments the first step 110 of encoding is configured to encode the chemical reaction graph into a line notation, the method further comprising, prior to the second step 120 of encoding, a step 130 of augmenting the line notation encoding.

The step 130 of augmenting is performed, for example, by a computing system configured to run a dedicated software. During this step 130 of augmenting, the line notation of the chemical reaction graph is reorganised so as not to change the nature of the chemical reaction encoded while still providing an alternative encoding for that chemical reaction.

An example the result of the step 130 augmenting is shown in FIG. 12.

In particular variants, the reaction can be reduced to the reaction site in a step (not represented) of reduction of a reaction encoding or chemical reaction graph. Such a step of reduction of a reaction encoding is performed, for example, upon a line notation resulting from the first step 110 of encoding or the second step 120 of encoding so as to remove all atom identifiers that remain inert during the chemical reaction modelled. An atom identifier is removed, for example, if said atom identifier and the associated bonds are located in a molecule that remains unchanged from reagent the reaction stage to the product stage of the chemical reactions.

This step of reduction of a reaction encoding further compresses the chemical reaction graph to the useful set of symbols. FIG. 6 shows a reaction 615 reduced to the reaction site for the formation of the products. The reaction is indicated including the first neighbouring atom.

In variants where the reaction, 615 in FIG. 6, is reduced to the reaction site, a linear notation limited to the atoms and bonds subject to modification during the chemical reaction result 620 may be obtained correspondingly. Such a linear notation may be labelled ‘SiteSMARTS’. Such a result may correspond to the output of the first step 110 of encoding or to the output of a dedicated step of reduction of a reaction encoding that may be positioned upstream or downstream of the first step 110 of encoding.

Indeed, in a reaction we can distinguish reagents, which are chemical compounds that interact between themselves to produce the products, as well as other chemicals, such as catalysers, solvents, which are not changing during the reaction. The atom mappings are thus required only for chemicals with changing bonding information while non-interacting/non-changing parts can be skipped.

A ‘SiteCRS’, corresponding to a compressed chemical reaction graph limited to the reaction site can be computed using the following steps:
a step of identification of the reaction site,
a step of flagging all site atoms as ‘relevant’ for the reaction, optionally including the neighbours up to a user-specified topological depth, and/or atoms in the atom's ring or ring system,
a step to remove all atoms not flagged ‘relevant’ from the molecule,
a step of export of the condensed graph of reaction with the reaction site to a character string, optionally canonical.

In current datasets, reactions can be written using the SMARTS format, i.e. ‘reagent>agent>product’. The SMARTS for the full reaction are converted to SiteSMARTS applying the following steps:
a step of identifying the atoms with changed environment based on atom and bond changes,
a step of flagging all atoms with changes as relevant, optionally including neighbours up to the user-specified topological depth, and/or atoms in the atom's ring or ring system,
a step of removing all atoms not flagged ‘relevant’ from the molecule,
a step of renumber the map numbers on the atoms by optional canonicalization and
a step of exporting SMARTS to create a SiteSMARTS, optionally canonical.

A subset of reactions is, for example, analysed from the NextMove Pistachio dataset 5. The reactions can be split and analysed by the published class, e.g. class 1.1.1 defines the Chan-Lam alkylamine coupling. The following steps can be, for example, applied:
a step of identifying the reagents and product involved in the net reaction,
a step of removing agents, solvents and uninvolved reactions,
a step of computing the SiteSMARTS and SiteCRS to characterise the reaction.

The SiteCRS generated can be used to cluster the reaction transformation using a string tag instead of a fingerprint. There are two main advantages: A chemist can understand the tag and thus check if the obtained tags are relevant for the type of reaction during the curation process.

The compressed chemical graph of reaction, according to the format resulting from the method 100 object of the present invention, can be extended to include changes for stereochemistry, e.g. {-/} and {-\} defines a change from a single bond to an upright or downright single bond and {-\hat{ }} et {-_} for single bonds changing to ‘single up’ or ‘single down’ for relative stereochemistry on a tetrahedral centre. It is equally possible to go from a double bond to a single up or single down, thus the symbols {=\hat{ }} and {=_} may be used. It is thus possible to go the reverse way, e.g. {\hat{ }=} from single up to double and {_=} from single down to double. An example of such a reaction is the hydrogenation of alkynes. Depending on reaction condition, the chemists can run a reaction with a syn- or anti-hydrogenation to make cis- and trans-alkenes from alkynes, respectively. An example of a stereochemical reaction with a tetrahedral stereocenter is the biocatalytic reduction of a ketone to a secondary alcohol by the enzyme class alcohol dehydrogenase. An example is the reduction of raspberry ketone to 4-3R-hydroxybutyl) phenol.

As a reminder, FIG. 2 shows an example of formatted chemical reaction graphs 205 and 210, obtained by the method 100 disclosed above.

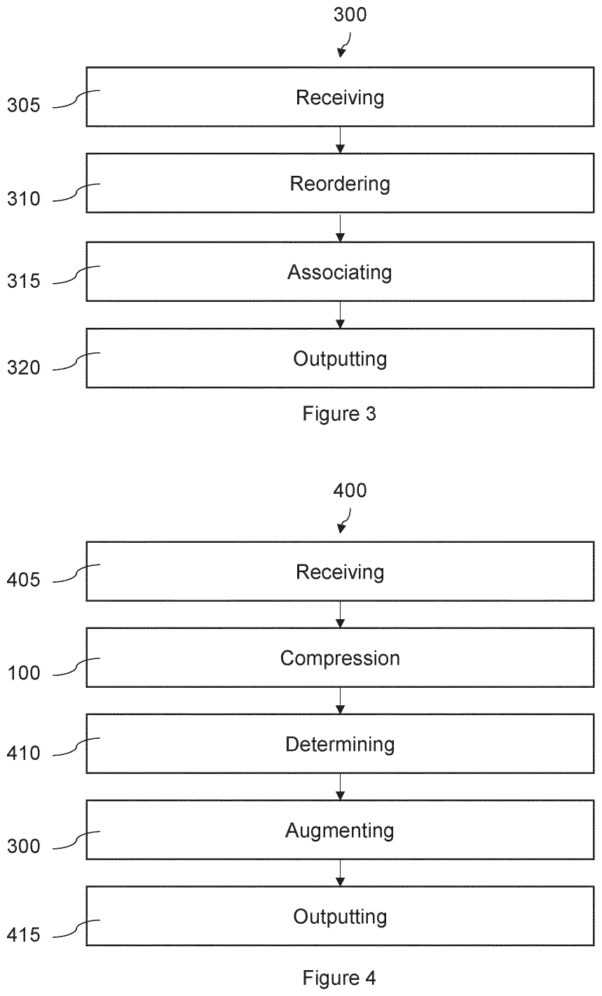

FIG. 3 shows a particular embodiment of the method 300 object of the present invention. This chemical reaction dataset augmentation method 300 comprises:
a step 305 of receiving, upon a computer interface, a string of characters according to the format resulting from any variant of the implementation of the method 100 disclosed with respect to FIG. 1,
a step 310 of reordering, by a computing system, the string of characters in order to shift at least one character representative of an atom and at least one string of at least one character representative of a change of bond associated to the corresponding atom and
a step 320 of outputting, upon a computer interface, an augmented string of characters corresponding to the reaction initially encoded by the received string of characters.

Functionally and structurally, the step 305 of receiving is analogous to any variant of the step 105 of receiving disclosed in regard of FIG. 1.

The step 310 of reordering is functionally and structurally similar to the step of augmenting 130 disclosed in regard of FIG. 1. During this step 310, the symbols or characters of a chemical reaction graph formatted and compressed according to the method 100 are formally reorganised to provide an alternative encoding representative of a single chemical reaction graph. Such an example can be seen in FIG. 2, in which a chemical reaction graph is formatted and compressed in two alternative encodings, 205 and 210.

In particular variants, the method 300 object of the present invention comprises a step 315 of associating, by a computing system, at least two strings of characters according to the format resulting from any variant of the implementation of the method 100 disclosed with respect to FIG. 1, each said string of characters being representative of the same chemical reaction graph.

This step 315 of associating is performed, for example, by a computing system configured to run a dedicated software. During this step 315 of associating, alternative compressed encodings for a chemical reaction graph may be concatenated into a single string of characters and preferably separated by a neutral symbol or character, such as a dot in the example 215 shown in FIG. 2.

The step 320 of outputting is functionally and structurally similar to the step of providing 125 disclosed in regard of FIG. 1.

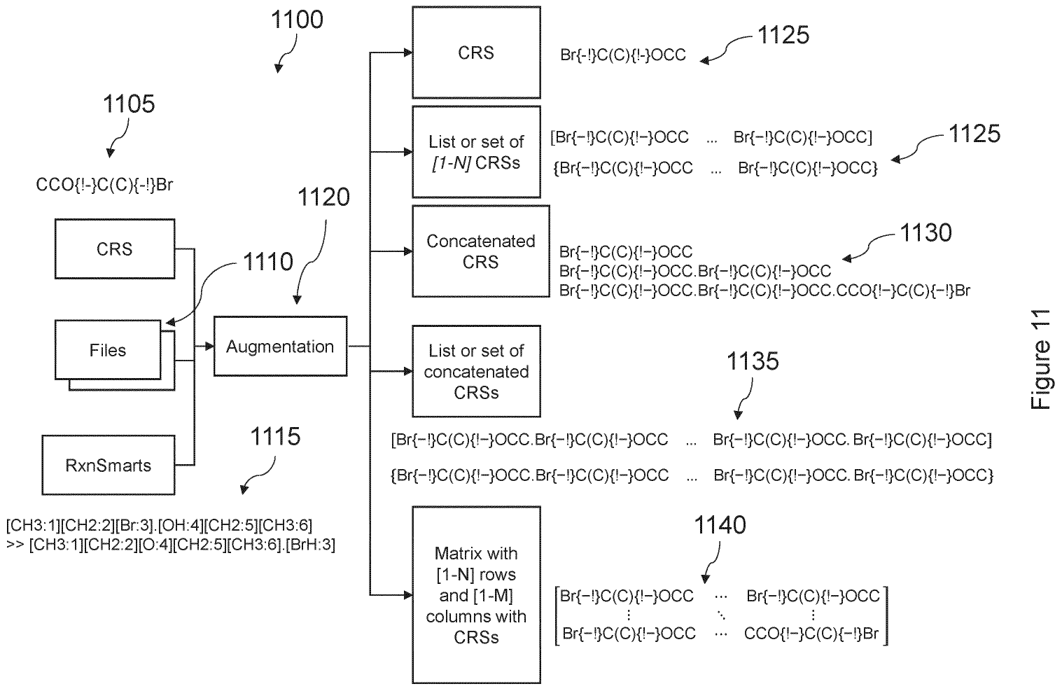

A broader view of the augmentation capabilities 1100 achievable by the use of the present invention can be seen in FIG. 11. FIG. 11 shows several possible augmentation inputs, 1105, 1110 and 1115, such as:
a compressed and formatted chemical reaction graph 1105 according to the format object of the present invention, such format being abbreviated CRS (for ‘chemical reaction string’),
an input (such as a file) 1110 defining one or more valid reagents and products in any machine-readable chemical format, including .mol (for ‘Molfile’), .sdf (for ‘Structure-data file’), .xyz (‘XYZ file format’) files for example and/or
an input representative of a line notation of a chemical reaction, such as a SMARTS encoded chemical reaction, such format being abbreviated RxnSmarts.

FIG. 11 shows several possible augmentation outputs, 1125, 1130, 1135, 1140 and 1145, such as:
an alternative compressed and formatted chemical reaction graph 1125 describing the same reaction with a change of atom order—a canonical form may be used to standardise the atom order,
a finite list of [1, N] compressed and formatted chemical reaction graphs defining the same reaction, this list being possibly reduced to a set of unique compressed and formatted chemical reaction graphs,
a finite list of [1, N] compressed and formatted chemical reaction graphs delimited, e.g. using the dot character ‘.’, compressed and formatted chemical reaction graphs describing the same reaction, which may be reduced to a set of unique compressed and formatted chemical reaction graphs,
a list or set of [1-N] finite lists of [1, N] delimited, compressed and formatted chemical reaction graphs,
a finite matrix with [1-N] rows and [1-M] columns defining single or concatenated compressed and formatted chemical reaction graphs for the same reaction and/or
a list or set of [1-N] finite matrices of [1-N] rows and [1-M] columns defining single or concatenated compressed and formatted chemical reaction graphs.

Such augmentations 1120 may be achieved similarly to the step 130 of augmentation or the step 310 of reordering such as disclosed above.

Augmenting a data may be used in a variety of applications:
data increase to learn models for small datasets,
equilibration of unbalanced datasets and/or
learning of a neural network or model using ensemble representation.

FIG. 4 shows a particular embodiment of the method 400 object of the present invention. This chemical reaction dataset preprocessing method 400 comprises:
a step 405 of receiving, upon a computer interface, a dataset of at least two chemical reaction graphs comprising at least one chemical reaction reagent and at least one chemical reaction product,
a step 100 of compression of at least two chemical reaction graphs according to the method disclosed in regard of FIG. 1,
a step 410 of determining, by a computing system, a distribution of chemical reaction classes within the encoded dataset,
a step 300 of augmenting the dataset, according to the method disclosed in regards of FIG. 3, for at least one chemical reaction class as a function of the determined distribution and
a step 415 of outputting, upon a computer interface, the preprocessed dataset.

Functionally and structurally, the step 405 of receiving is analogous to any variant of the step 105 of receiving disclosed in regard of FIG. 1. This step 405 of receiving can be performed by implementing several successive or serial instances of the step 105 of receiving or by implementing one single step 105 of receiving configured to receive, in one input, the several datasets.

The step or method 100 of compression is disclosed, in several variants, in regards of FIG. 1.

The step 410 of determining is performed, for example, by a computing system configured to run a dedicated software. During this step 410 of determining, statistical analysis is performed upon the dataset and compared to a static or dynamic threshold of acceptability. Such a threshold may be, for example, in terms of samples per reaction class in absolute or relative value, with regards to the sample for other reaction classes in the dataset. The terms ‘chemical reaction class’ are also referred to as ‘chemical reaction type’ (such as synthesis, decomposition and replacement).

The step or method 300 of augmenting the dataset is disclosed, in several variants, in regards of FIG. 3. Alternatively, this step 300 of augmenting the dataset may instead or in parallel rely upon the implementation of the step 130 of augmenting the dataset prior to the second step 120 of encoding to augment the dataset.

The step 415 of outputting is functionally and structurally similar to the step of providing 125 disclosed in regard of FIG. 1.

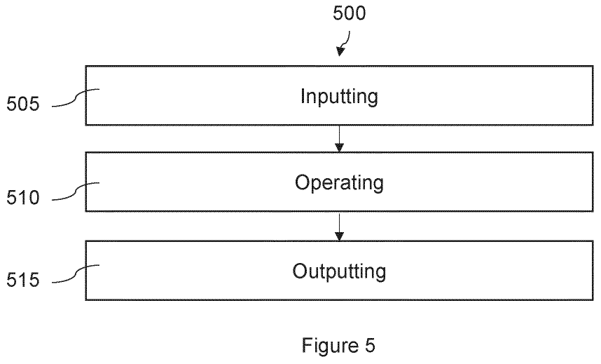

FIG. 5 shows a particular embodiment of the method 500 object of the present invention. This training method 500 for a classifier, transformer or regressor comprises:
a step 505 of inputting, upon a computer interface, a dataset of chemical reaction graphs encoded in the compressed format such as obtained by any variant of the method 100 disclosed in regards of FIG. 1,
a step 510 of operating, by a computing system, a recursive neural network architecture configured to use, as input, the dataset of chemical reaction graphs to classify the chemical reaction bond evolution as a function of the input and
a step 515 of outputting, upon a computer interface, a trained classifier, transformer or regressor.

Functionally and structurally, the step 505 of receiving is analogous to any variant of the step 105 of receiving disclosed in regard of FIG. 1. This step 405 of receiving can be performed by implementing several successive or serial instances of the step 105 of receiving or by implementing one single step 105 of receiving configured to receive, in one input, the several datasets.

The step 510 of operating is performed, for example, by running a recursive neural network architecture and associated software upon a computing system, based upon a training set.

The step 515 of outputting is functionally and structurally similar to the step of providing 125 disclosed in regard of FIG. 1.

Regarding regressors may be trained according to the targets ‘reaction yield’, ‘equilibrium constant of the reaction’ or ‘transition state energy’.

Such a regressor may be trained according to any of the following examples:
‘Predicting reaction performance in C—N cross-coupling using machine learning’ by D. T. Ahneman, J. G. Estrada, S. Lin, S. D. Dreher, A. G. Doyle—Apr. 13, 2018, or
Schwaller, Philippe; Vaucher, Alain C.; Laino, Teodoro; Reymond, Jean-Louis (2020): Prediction of Chemical Reaction Yields using Deep Learning. ChemRxiv. Preprint. (https://doi.org/10.26434/chemrxiv.12758474.v2).

The present invention also aims at a chemical reaction bond evolution prediction method, operating a classifier, transformer or regressor obtained by the training method disclosed in regards of FIG. 5.

These embodiments allow for the detection of the sites of reaction. Such embodiments have been disclosed above.

The present invention also aims at a chemical reaction generation method, operating a classifier, transformer or regressor obtained by the training method disclosed in regards of FIG. 5.

Such a chemical reaction generation method uses, as input:
a compressed and formatted chemical reaction graph obtained by the method 100 object of the present invention, which is tokenized to a vector of length N containing a discrete value to identify the type of character in part of the compressed and formatted chemical reaction graph, such as a one-hot encoder,
a compressed and formatted chemical reaction graph obtained by the method 100 object of the present invention which is tokenized to a one-hot encoded matrix of dimensions N×M comprising N rows defining the possible characters and M columns describing the length of a part of the compressed and formatted chemical reaction graph,
a one-hot encoded vector of size M defining the next character,
flexible reaction bonds in the compressed and formatted chemical reaction graph obtained by the method 100 object of the present invention can optionally be used as a character group, e.g. ‘{!-}’ defines single position in the vector and/or
the tokenizer adding a stop character at the end of the sentence.

Such a chemical reaction generation method uses, for example, as a network, a four-layer architecture comprising:
an input layer taking a tokenized vector or matrix for sequence length N and M possible characters,
one or more recursive neural network (RNN) of sequence length from 2 to 1024,
a dropout layer of a fraction (from 0 to less than 100%) of the output of the RNN and a dense layer of a vector of size M with a probability for the next character.

Such a model can be trained the next most likely character in the network to be chemically correct. The network predicts the probability on all possible characters and selects the next character randomly. The writing is a recursive process of writing: Select—Predict—Select—Predict until a finite number N of valid reactions has been produced.

The output of the network is sequentially written CRSs within or without the learned reaction space depending on how deeply the generative model is trained.

FIG. 12 further shows an architecture 1200 executing the two steps of training 1205 a generative neural network and generating 1210 reactions as well as associated steps of inputting 1215 sample data to train the generative neural network and outputting 1220 the generated reactions.

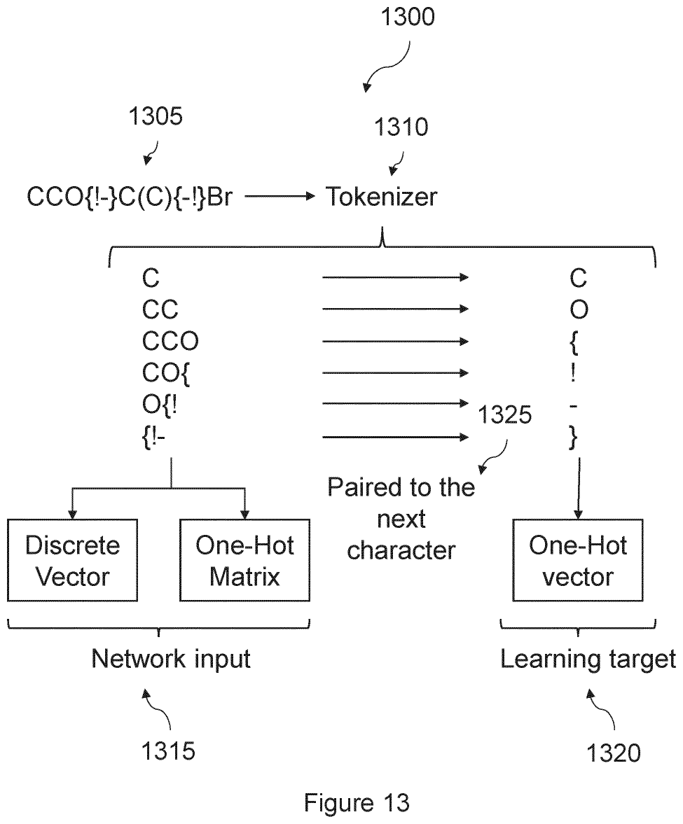

FIG. 13 further shows the training method 1300 disclosed above in which:
compressed and formatted (encoded) chemical reaction graphs are input 1305 into a tokenizer 1310 and
said tokenizer 1310 being configured to operate:
   a step of tokenizing the compressed and formatted (encoded) chemical reaction graphs into a network input 1315, said network input 1315 being of either discrete vector or one-hot matrix type and
   a step of pairing 1325 each token with the following character in the input compressed and formatted (encoded) chemical reaction graph, said tokens being used as a learning target 1320 for the RNN, said learning target 1320 being organised, for example, in a one-hot vector.

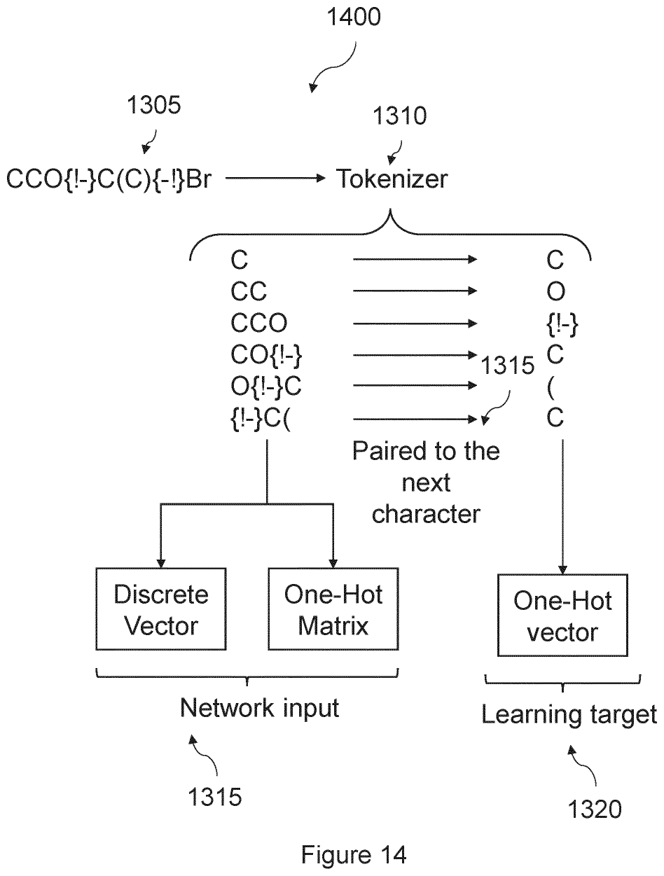

FIG. 14 shows an alternative 1400 to FIG. 13 in which the string of characters encoding a change of bonds between atoms is encoded as a specific unitary token.

FIG. 6 shows a particular embodiment of the states 600 of encoding of a chemical reaction via the software object of the present invention.

For example, a chemical reaction graph can be seen in FIG. 6, with regards to references 605 and 610. FIG. 6 shows the Williamson ether synthesis as an example. Reference 605 designates ether synthesis between ethyl alcohol and ethyl bromide to form diethyl ether and 610 designates ether synthesis between cyclohexanol and ethyl bromide to form ethoxycyclohexane.

FIG. 7 shows a particular embodiment of the states 700 of encoding of an equilibrium chemical reaction via the software object of the present invention. These states 700 comprise:
the equilibrium reaction 705, with reaction constant K, showing full atom mapping and the net chemical equilibrium,
the SMARTS and compressed chemical reaction graph for the forward reaction 710 and
the SMARTS and compressed chemical reaction graph for the backward reaction 715.

The compressed and formatted bonds between the forward and backward reactions, indicate the easy reversibility of the format object of the present invention by changing the bond order in the string of characters. This can easily be seen in the ‘=’ and ‘!’ character swap shown between the reactions 710 and 715 representing reverse actions such as synthesis and retrosynthesis. This greatly reduces the amount of data to be stored and the capacity to use fewer sample for machine learning applications.

For example, any reaction such as the reaction 705 can be formally represented by an equilibrium where the constant K can define the ratios between products and reagents. The value of K can vary from zero to infinity. The phenomenon can be used to augment reaction data by using both CRS representations (preferably, combining forward and backward reaction CRSs).

An additional and important advantage of the format of the present invention is the compressibility for large datasets. Compressed chemical reaction graphs define the shortest format to define a net chemical reaction available today.

FIG. 7 also shows the capacity of the format to add reaction conditions, such as solvents and/or catalysts for example, to the CRS character string. The example shown here is the Grignard reaction, which is performed using magnesium Mg in the solvent diethyl ether. Water, chemically written as ‘O’ in a CRS, is used to stop the reaction by hydrolysis. This type of CRS can be considered as a ‘conditional CRS’ to propose reaction conditions for a given CRS.

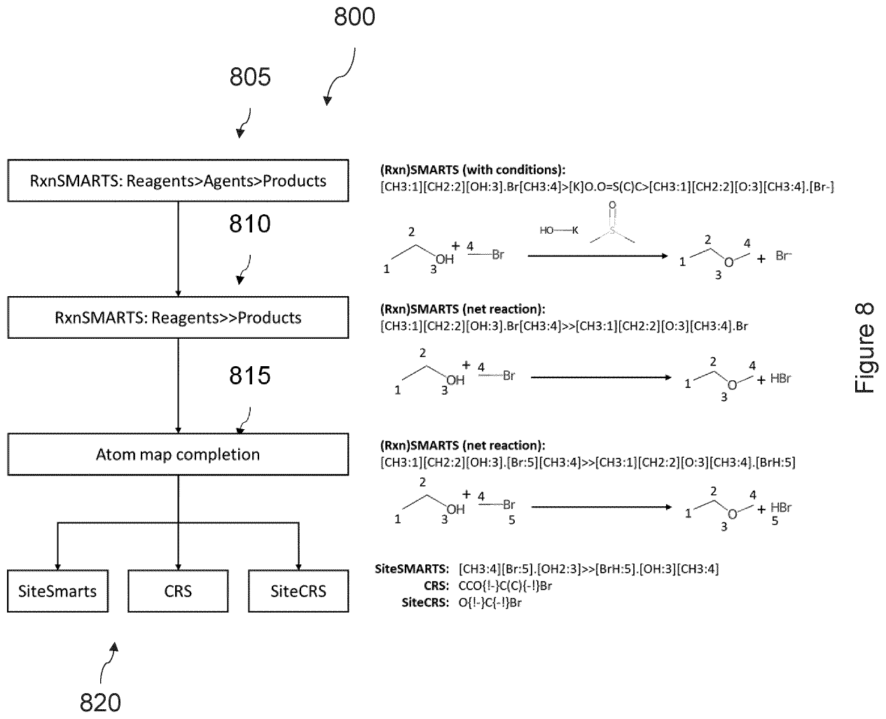

FIG. 8 shows, schematically, instructions of a particular embodiment 800 of the software object of the present invention. These instructions are:
inputting 805 the RxnSMARTS format, for example including alkaline conditions (KOH) and solvent (Me2SO)
cleaning 810 the reaction RxnSMARTS with involved reagents and products only; this step also neutralises the reagents and/or products and defines the net chemical transformation,
completing 815 of the atom map numbers to define the full net chemical reaction and
producing 820 CRS, SiteCRS and/or SiteSMARTS.

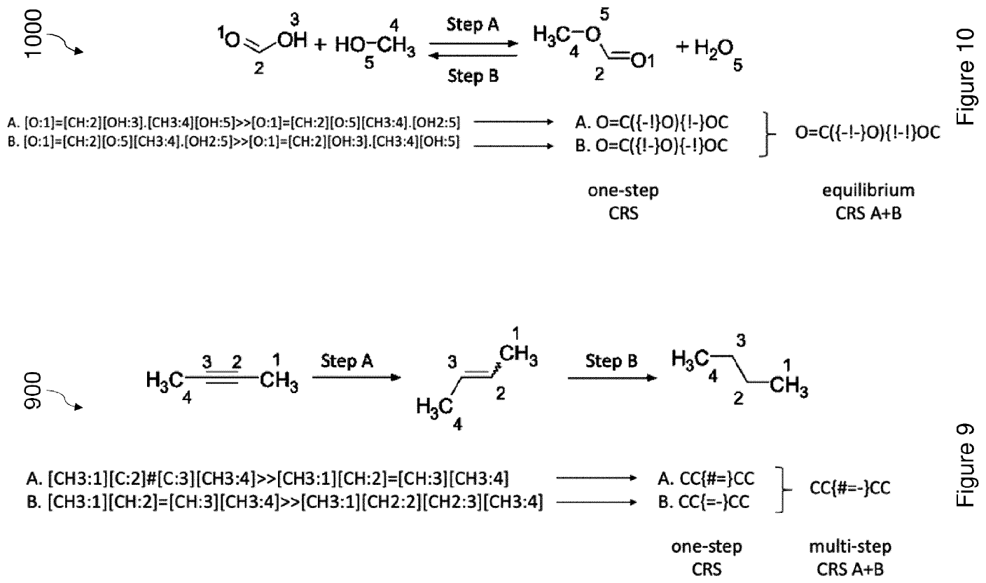

FIG. 9 shows, schematically, successive reactions steps (A and B) encoded within a multistep reaction encoding 900 by the software object of the present invention.

In particular embodiments, multistep reactions, represented by a succession of change of bonds between two atoms, are encoded by a succession of single characters, each single character being representative of the successive state of a bond between said two atoms, the order of the characters being representative of the order of changes of bonds between said two atoms.

In such embodiments, the change of bonds between two atoms are encoded this way: ‘Atom symbol 1’ ‘{’(neutral character) ‘Reagent bond character’ ‘Product of the first step of reaction bond character’ ‘Product of the second step of reaction bond character’ ‘Product of the n-th step of reaction bond character’ ‘}’ (neutral character) ‘Atom symbol 2’.

FIG. 10 shows, schematically, equilibrium reactions encoded within an equilibrium reaction encoding 1000 by the software object of the present invention.

The new reaction format disclosed herein is the shortest possible syntax to write a net chemical transformation. Indeed, the newly produced compressed chemical reaction graph has a length of approximately 20% when compared to the corresponding RxnSMARTS for the same reaction (FIGS. 6 to 10).

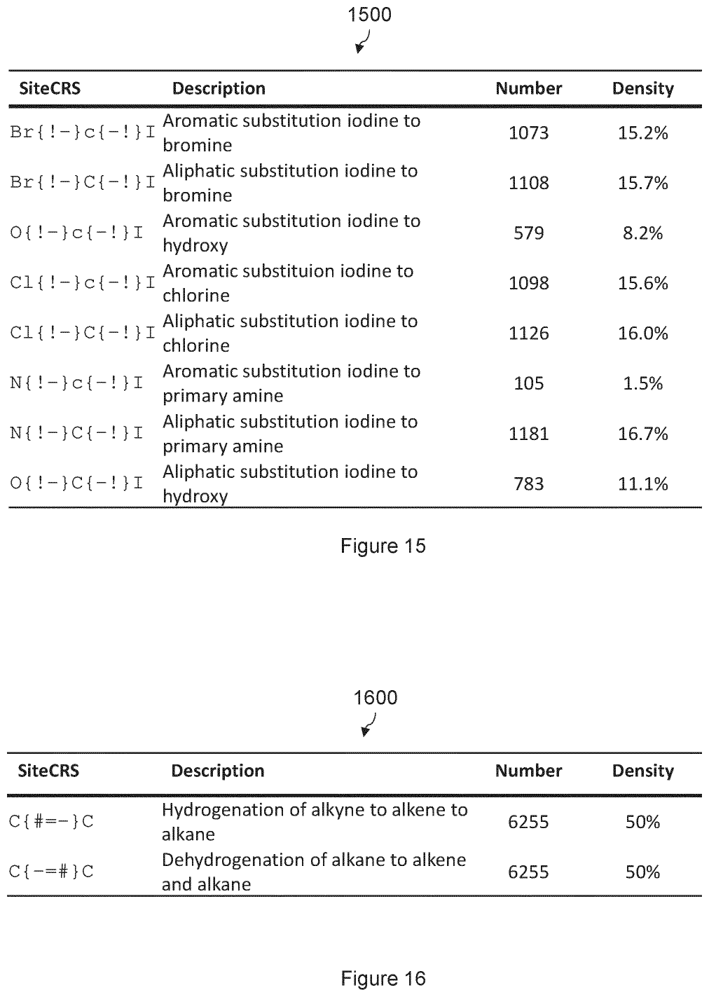

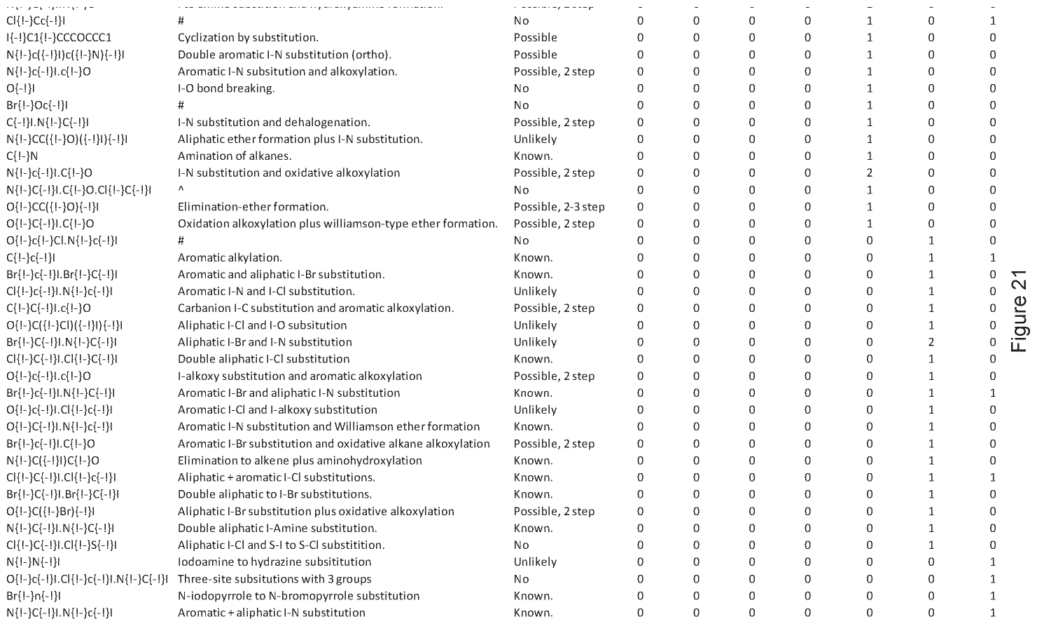

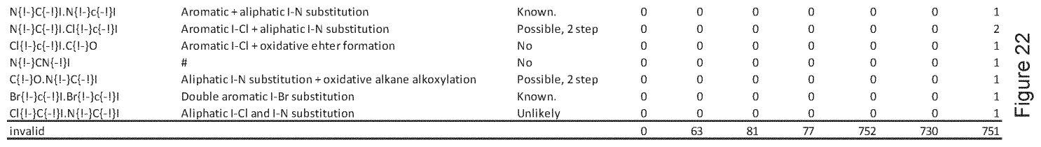

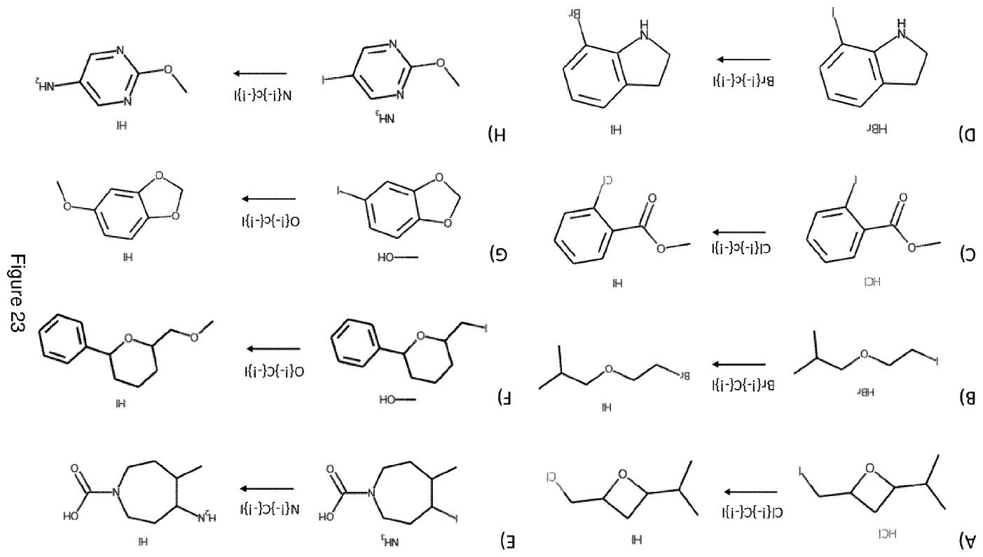

Such a chemical reaction generation method may also be understood from the perspective of FIGS. 15 to 27.

In recent years, generative neural networks have become powerful deep learning methods to generate realistic in silico data from real-world examples. Generative neural networks have been successfully used to generate deep fakes for images and voice to make realistic computer-generated images and movies. Examples of deep generative models include variational auto-encoders (VAE) which are based on sampling from a latent space, typically Z (μ, σ²), with a set of compressed parameters or using generative adversarial network in which two networks, i.e. a generator G and a discriminator D, iteratively compete to generate realistic synthetic solutions that can no longer be differentiated from the real data by the discriminator.

Within chemistry, generative models are highly useful for molecule discovery using the above technology to generate new molecules. In particular, generative neural networks that have learned to write the chemical language SMILES as is have been used using methodology known from natural language processing. These approaches are limited to molecular level processing. This invention proposes to include an examination mechanism by stochastic sampling. This new strategy introduced generative examination network defining an adaptation of the early-stopping function to maintain the highest level of creativity. In this examination mechanism, the model generates a statistical sample of reasonable size to evaluate the models' success on writing chemically correct SMILES strings, i.e. SMILES that can be processed by chemical toolkits without errors. As exemplified, the training of the neural network is stopped after the network is statistically stable on the generated entries.

The format object of the present invention provides syntax to define a one-line notation of chemical reaction graph. This syntax, which can, in no limiting manner, be referred to as ‘Chemical Reaction String’ (CRS) introduces reaction bonds to line notations. The syntax stands apart because it defines a large compression of the currently known reaction SMARTS and does not require any explicit atom indexing. A CRS may be extended including auxiliary unmodified molecules. The CRS syntax includes two major benefits: 1) easy reversibility of the reaction by inversion of the used bond symbols; 2) easy extension for multi-step reactions by adding additional steps to the flexible bonds. Herein these capacities are exemplified for the following set of reactions: 1) A set of eight substitution reaction with iodine as leaving the group. 2) Multistep hydrogenation and dehydrogenation between alkynes, alkenes and alkanes. Lastly, a major advantage of generating a single step or multi-step reaction with CRS strings is the immediate production of multiple tasks. First, any single reaction CRS defines simultaneously the reagents, the products and the reaction. Second, it is thus possible to include conditions to a reaction, e.g. an unchanging molecule or solvent. Example: ‘CC ({-!}Br) C(C) {={=-} O.CCOCC. [Mg]. O′ for a Grignard reaction. In this string CCOCC and [Mg] are auxiliary reagents.

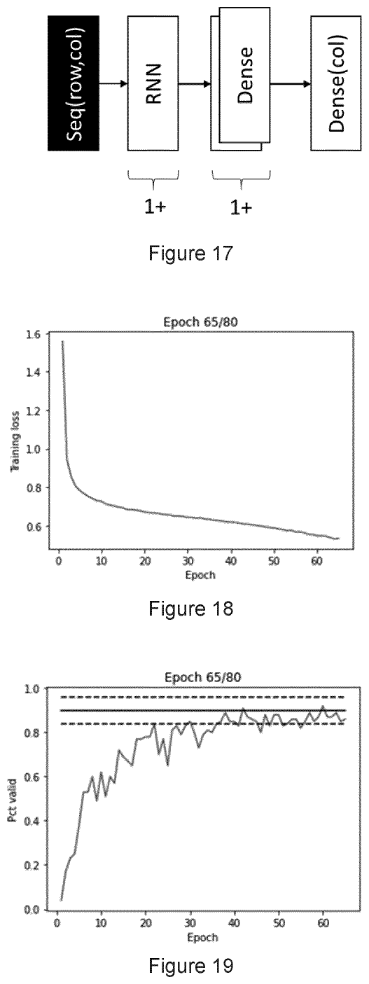

One such example makes use of the following technical considerations:
datasets: In this work generated datasets using molecules published in PubChem were used. Subsequent generated reaction datasets based on well-known reactions were obtained. For an example of single-step reaction substitution reactions on the strong leaving group iodine were used. For multistep reactions, hydrogen of alkynes via alkenes to alkanes was used.
substitution reactions: From PubChem were selected aliphatic and aromatic iodine molecules with a single iodine. Eight substitutions on the iodine were applied, defining eight different single step substitution reactions (FIG. 15). In these reactions 1500, iodine is the stronger leaving group, and the reaction are considered a non-equilibrium reaction.
hydrogenation reactions: From PubChem were selected molecules with a single aliphatic carbon-carbon triple bond. This bond was converted in a multistep reaction to define the reaction 1600 alkyne>alkene>alkane (FIG. 16). All forward reactions were inverted by replacing the hydrogenation bond, i.e. {#=−}, into a multistep dehydrogenation bond, i.e. {-=#}.
neural networks: in this example recursive neural networks were used to predict the next possible character. Such a network defines thus an iterative writer, sampling the next possible characters based on the sequence of previously written characters. The neural network used herein is composed of the following layers (FIG. 17):

| Layer | Parameter | Descriptors |
|---|---|---|
| Input | [row, col] | One-hot encoded matrix with size [row, col]. Row |
| | | defines the length of the analysed sequence; col |
| | | defines the number of known tokens in the library. |
| RNN | [2, max length] | One or multiple RNN layers to evaluate the |
| | | sequence. Max length corresponds to the |
| | | maximum sequence length. |
| Dense | L = [2, 1024] | One or multiple dense layers with size L and |
| | A = [‘relu’, ‘srelu’, | activation A. |
| | ‘sigmoid’, ‘tanh’, | |
| | ‘leakyrelu’, ‘softmax’] | |
| Dense | col | Dense output layer with length col and softmax |
| | | activation to predict the probabilities for the next |
| | | characters. |

The example neural network is trained using a categorical cross-entropy. The training of the neural network was stopped by using an examination mechanism. The examination mechanism is an early-stopping function that generates a statistically relevant sample of tens or hundreds of generated entries and measures the number of valid entries. The early stopping function stops training when the model shows statistically stable results based on a user-specified percentage of valid entries. The percentage of valid entries is considered statistically stable, when the percentages stay within the 90% confidence interval for the used sample size for a minimum of 10 epochs. The generator mechanism as written below has also been used as a generator for this early-stopping function.
The neural network used for generation is used herein to predict the next possible character based on the previously written characters. The network is thus an iterative writer. FIG. 17 shows the network layout. The network used herein to exemplify the application is a network taken a one-hot encoder matrix describing the sequence as input. FIG. 18 shows a monitoring plot of the learning process showing the development of the categorical cross-entropy loss function. FIG. 19 shows an early stopping function used in the generative examination network showing the percentage of valid reactions generated by the generative neural network. The bold and dashed line show the mean percentage with the associated 90% confidence interval for a sample size of 100 generated reactions. Training is stopped early if the result was statistically stable within the 90% confidence interval. In the example above, the training was thus stopped after 65 epochs.
Generation: upon completion of the training of the neural network, i.e. when the neural network has obtained a statistical stable result for the generation of valid reactions, the generation process is started. The generator is an iterative writer, predicting the next possible character based on the last number ‘n’ of characters. If fewer characters were written, the generator uses all characters. The initial seed used is ‘\n’ to define the end of the previous molecule. During generation, the method iteratively writes characters, e.g., ‘\nC’, ‘\nCC’, ‘\nCCC’, etc. Once the size n+1 has been reached, the method uses only the last n characters of the word to predict the next characters.
Evaluation: the model evaluation for a set of 180 reactions is performed by counting the number of correct reactions and extracting the SiteCRS, i.e. the key for the reaction site defining the reaction type. Based on the results, it is evaluated whether some reactions are generated more frequently, less frequently or with the approximate ratios than the ratios in the dataset. For this calculation, the number of invalid reactions has been neglected in the computation and have been listed separately in the table.

Such an example yielded the results disclosed below.

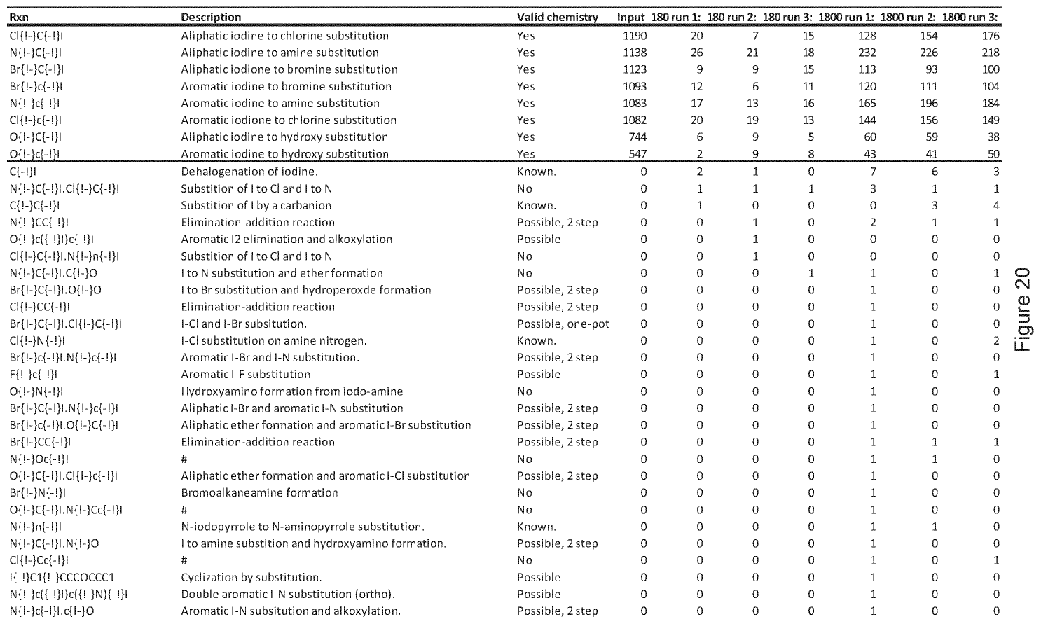

FIGS. 20 to 22 show generation results for the substitution reaction. Reactions flagged with (#) defined readable but invalid reactions for reasons of valency errors. Reactions flagged with (\hat{ }) are reactions composed of a mix of multiple type of substitutions. The word ‘known’ reverse to a reaction known from literature. The word ‘possible’ indicates the reactions might be possible. The word possible has been commented with ‘2 step’ to indicate that the reaction is probably composed of two independent steps and ‘one-pot’ indicating that both steps can be performed in one-step, even though the reactions are different.

FIG. 23 shows the generated examples for the input reactions. The reactions as proposed by the generator define a reaction for a valid reagent and a valid product. The generator was exclusively trained with the knowledge to postulate possible chemical reactions and was not trained with information on yield. A) Aliphatic iodine to chlorine substitution. B) Aliphatic iodine to bromine substitution. C) Aromatic iodine to chlorine substitution. D) Aromatic iodine to bromine substitution. E) Aliphatic iodine to amine substitution. F) Methyl ether formation using a Williamson-type reaction. G) Aromatic methoxylation by substitution of iodine. H) Aromatic substitution of iodine to primary amine.

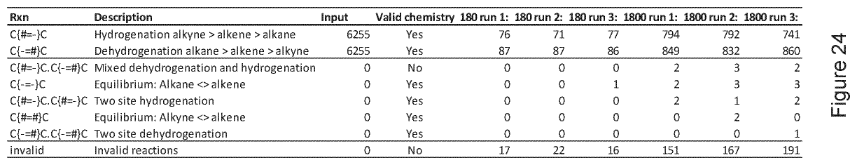

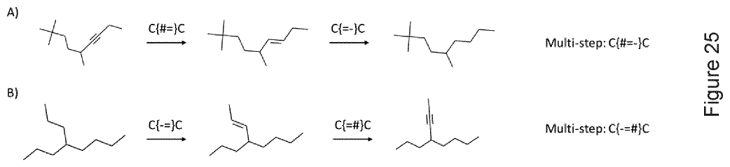

FIG. 24 shows the results for the multi-step hydrogenation and dehydrogenation.

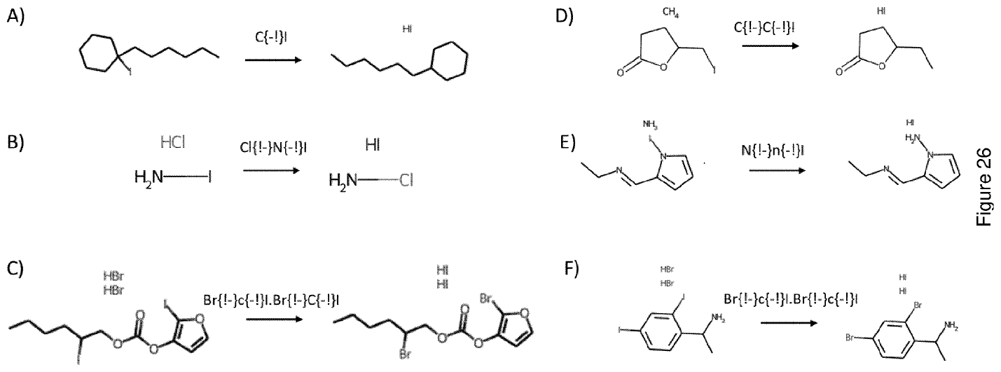

FIG. 25 shows examples of generated reactions for the input reactions of the model. All reactions have been produced as a multi-step reaction using the SiteCRS indicates on the right. For exemplification, the multi-step has been decomposed in its first and second step. The reactions displayed here are generated in silico and have not been evaluated for synthetic feasibility.

As it can be understood, the generation method object of the present invention creates a single reaction dataset composed of 8 different substitution reactions on aromatic and aliphatic iodines. All substitutions have in common that the strong leaving group iodine is replaced by another nucleophile. In the results, it can be seen that the reaction generator is capable of generator examples for all reactions available in the training set. Note, all percentages of the valid reactions have been computed excluding the number of invalid reactions. Consequently, the displayed density values can be compared to the reaction densities in the input set. We observe in all samples that the density in the generated set may vary significantly from the densities in the input set. Nevertheless, the majority of the reactions fall within the class of reactions presented to the generative neural network. The statistical variation is clearly an important advantage of this generative neural network: the generator is free to generate based on selection the next character within the bounds of the predicted probability. As a consequence, the distribution on generated reactions may vary between a set of generated molecules. Additionally, the freedom of the generator is an important advantage for the creation of new reactions. These new reactions include the substitutions at multiple sites but sometimes the reactions define new ideas that were previously unknown to the generator. An example of such a reaction if the N-iodopyrrole to N-aminopyrrole substitution. This example is remarkable because the input dataset only contained substitutions on carbon atoms. In summary, the reaction generator can thus propose both reactions within the same reaction space as well as generate new reaction based on the acquired knowledge of writing chemically correct molecules. An essential mechanism to maintain the creativity of the generator is the use of a stochastic examination mechanism that periodically tests the knowledge of the generator to create valid chemical reactions.

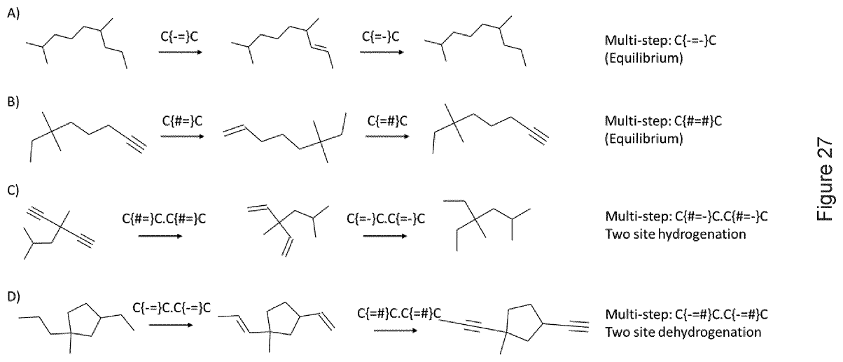

FIG. 26 shows examples of new reactions created by the generator. Albeit unknown in the input set, i.e. it was composed of the 8 reactions initially defined, the generator has generated new reactions. All reactions are shown with reagent, product and the SiteCRS above the reaction arrow. The examples are: A) Dehalogenation of an alkane. B) Iodine to chlorine substitution on an amine. C) Aliphatic plus aromatic iodine to bromine substitution. D) Substitution of iodine by a carbanion. E) Substitution of N-iodopyrrole to N-aminopyrrole. F) Double aromatic substitution from iodine to bromine.

As an example of multistep reactions, a generator was trained with multi-step hydrogenation, i.e. from alkyne to alkene and from the produced alkene to alkane. Within the dataset was also defined the dehydrogenation as a multistep reaction, i.e. alkane to alkene and the alkyne. The hydrogenation and dehydrogenation are thus written with the SiteCRSs C {#=-} C and C {-=#}, respectively. The CRS syntax has been chosen to be flexible and capable of accommodating multiple reaction steps. The SiteCRS for the multistep hydrogenation, i.e. C {#=—} C is an inner join of two hydrogenation reactions: 1) alkyne to alkane written as C {#-} C and 2) alkene to alkane written as C {=-}. The example of a two-step reaction is the first expansion of the previously shown single reactions. At the user's discretion, this flexible bond type can be extended to include additional characters to define a third, fourth, etc. reaction. In comparison to the previous results, it can be seen clearly that the generator for these reactions has a higher success rate of generating valid reactions, even though the reaction generator had to take a multi-step reaction into account. The primary difference is the reduced diversity in the set of molecules, i.e. all molecules used in this dataset are aliphatic alkanes, alkenes and alkynes, whereas the substitution dataset includes both aromatic and aliphatic compounds. FIG. 24 summarises the generation results for 3 runs of 180 generated reactions and 3 runs of 180 generated examples. The reduced diversity in this set of molecules is also visible with a reduced level of creativity. Indeed, the set of proposed new reactions is very limited. Nevertheless, the generator shows the creation of new chemistry and has hypothesised new reactions. Firstly, it can be seen that the generator has generated molecules with multiple reaction sites, e.g. ‘{#=-}. {#=-}’ defining a molecule with two triple bonds (FIG. 27 C to D). This is remarkable because the model was trained with a dataset composed of a single site. Secondly, the generator has introduced equilibrium reactions, e.g. ‘C {-=-} C’ and ‘C{#=#}C’. These SiteCRSs define equilibrium reactions for the dehydrogenation of alkane to alkene and the hydrogenation of alkyne to alkene (FIG. 27A to B). The network is open to accommodate any type of one-step, two-step or multi-step reaction. An equilibrium as generated by the neural network (FIG. 27A to B) is a special type of a two-step reaction and can thus be dealt with using the CRS format.

FIG. 27 shows new reactions generation for the multi-step reactions. The above reactions are not presented in the dataset. The example includes the creation of two equilibrium reactions (A and B) and the creation of two multi-step reactions, even though the training only contained single reactions. A) Equilibrium for the alkane-alkene dehydrogenation. B) Equilibrium for the alkyne-alkene hydrogenation. C) Two-site hydrogenation reaction. D) Two site dehydrogenation reaction.

In other embodiments targeting the generation of reactions, an AI algorithm may be set up for the mining of chemical space which, to maintain diversity, introduces a statistical examination mechanism to select the earliest possible stage of the model that is reliably writing chemistry. The same algorithm can be applied to produce reactions, such as disclosed above.

The main advantage of a generated ‘CRS’ includes: 1) A product which can be extracted from the produced CRS; 2) reagents which can be extracted from the produced CRS. The route produced can then be looked up if the route is possible from existing starting materials.

The major benefit is thus that the product and the route are produced by a single generation. The possibility to generate a reaction rather than a single molecule is very different from the current approach. In the current approach: 1) We generate/define a molecule; 2) We think about a possible synthesis.

In the application ‘Generation’ we have CRS generation. We must defend the patent with this application and we may have to think about an additional application patent as a backup: We have shown that CPU computers rapidly mine the chemical space and that mining of the chemical space is an indispensable tool to identify new molecules (public data sources such as PubChem provide very few candidate molecules).

In other embodiments targeting the applications for regression and classification, the applications defined below apply to both the prediction of molecules themselves as well as to a produced CRS string. The CRS defines a reaction producing molecules of interest. Consequently, any predictive target for a molecule is also interesting for a prediction on a CRS, such as:
Regression/Classification for renewable carbon: from the proposed reactions the algorithm will tell whether the route is a route of renewable carbon. A product is said ‘renewable’ if all starting materials used are ‘renewable’. The higher the content, the better the future acceptance.
Regression/Classification for enzymatic reaction: From the reaction one can predict by regression/classification if the reaction can be an enzymatic reaction. The benefit of enzymatic reaction is that the product is considered ‘natural’. This will also push the future acceptance.
Regression/Classification on reaction yield: Can we roughly estimate whether a reaction works—even in the case that reaction yields are not remotely reported.
Regression/Classification on thermodynamic properties and transition state: Such a prediction is an energy prediction which can be beneficial to identify the ease of synthesis or the yield of a synthesis.
Regression/Classification on relevant targets for olfaction or taste: For the produced products one may be able to identify the following: 1) Whether the product may be introduced to the market (‘evaluation fate’); 2) olfactive descriptors; 3) relevant sensory and physico-chemical properties such as the odour detection threshold, odour value, henry, solubility, log P, volatility and/or vapour pressure; 4) Activity for olfactive receptors; 4) taste receptor activities (e.g. allosteric modulators that enhance sweetness); 5) top-heart-base note classification: this is a metric defining the strength. The mechanisms for the prediction can vary and may include knowledge-based methods in cheminformatics, classical machine learning methods and deep learning methods.
Regression/Classification of MS or NMR spectra: Predicting MS and NMR spectra may be used to confirm the identity for a new molecule.
Regression/Classification on predicting impurities: Such an application can help to anticipate on impurities produced by the reaction and in which quantities. Here we primarily think about mixture of stereo- (e.g. R-limonene or S-limonene) and regioisomers (para-Lyral and meta-Lyral). However, a predictive algorithm may also anticipate at other impurities produced.
Regression/Classification on predicting hazards: Here one needs to evaluate the stability in product, any type of toxicity, any type of accumulation (soil, water, . . . ).
Regression/Classification on production costs: This method looks at producing reactions.
Regression/classification of changing bonds: Predict the changing bonds on a product to get a reaction prediction (SMILES in=>CRS out). Such a prediction can possibly be reinforced with a quantitative reward for any of the properties (reinforcement learning): 1) ingredient on the market, 2) renewable carbon, 3) enzymatic reaction or 4) high-yielding reaction. In reinforcement learning one gives a reward for a solution that is particularly good because it satisfies some selection criteria.

As it is understood any embodiment may be used to encode, classify or generate any one of the non-limitative list of chemical reactions:

| NEXTMOVE CODE | NAME | | |
|---|---|---|---|
| | | c_2.1.2 | Carboxylic acid + amine condensation |
| | | c_6.1.1 | N-Boc deprotection |
| c_1.3.7 | Chloro N-arylation | c_9.7.33 | Carboxy to carbamoyl |
| c_2.1.1 | Amide Schotten-Baumann | c_3.1.6 | Chloro Suzuki-type coupling |
| c_6.2.2 | CO2H—Me deprotection | c_6.3.2 | O-TBS deprotection |
| c_7.1.1 | Nitro to amino | c_10.4.2 | Methylation |
| c_1.6.2 | Bromo N-alkylation | c_10.1.4 | Iodination |
| c_1.7.9 | Williamson ether synthesis | c_6.1.5 | N-Bn deprotection |
| c_6.2.1 | CO2H—Et deprotection | c_1.2.14 | Epoxide + amine coupling |
| c_3.1.1 | Bromo Suzuki coupling | c_3.3.2 | Bromo Sonogashira coupling |
| c_1.7.11 | SNAr ether synthesis | c_7.3.1 | Nitrile reduction |
| c_2.2.3 | Sulfonamide Schotten-Baumann | c_7.9.2 | Carboxylic acid to alcohol reduction |
| c_1.2.1 | Aldehyde reductive amination | c_1.3.2 | Chloro Buchwald-Hartwig amination |
| c_1.6.4 | Chloro N-alkylation | c_1.6.8 | Iodo N-alkylation |
| c_11.9 | Separation | c_2.1.7 | N-Acetylation |
| c_10.1.1 | Bromination | c_6.3.6 | O—Ac deprotection |
| c_3.1.5 | Bromo Suzuki-type coupling | c_7.2.1 | Amide to amine reduction |
| c_7.6.1 | Alkene hydrogenation | c_7.9.1 | Aldehyde to alcohol reduction |
| c_2.1.10 | Carboxylic ester + amine reaction | c_9.4.1 | Cyano to carboxy |
| c_5.1.1 | N—Boc protection | c_5.3.2 | O-TBS protection |
| c_1.3.8 | Fluoro N-arylation | c_8.2.1 | Sulfanyl to sulfinyl |
| c_6.3.7 | Methoxy to hydroxy | c_3.11.34 | Knoevenagel condensation |
| c_7.4.1 | Ester to alcohol reduction | c_1.6.9 | Mesyloxy N-alkylation |
| c_6.3.1 | O-Bn deprotection | c_1.1.4 | N-methylation |
| c_1.3.6 | Bromo N-arylation | c_3.3.4 | Iodo Sonogashira coupling |
| c_3.1.2 | Chloro Suzuki coupling | c_1.8.13 | Chloro thioether synthesis |
| c_1.7.7 | Mitsunobu aryl ether synthesis | c_10.1.5 | Wohl-Ziegler bromination |
| c_2.7.2 | Sulfonic ester Schotten-Baumann | c_2.6.3 | Fischer-Speier esterification |
| c_1.2.5 | Ketone reductive amination | c_3.8.1 | Wittig olefination |
| c_2.3.1 | Isocyanate + amine urea coupling | c_4.1.45 | Benzimidazole synthesis |
| c_2.6.2 | Esterification | c_1.7.5 | Hydroxy to triflyloxy |
| c_9.1.6 | Hydroxy to chloro | c_1.8.12 | Bromo thioether synthesis |
| c_7.5.1 | Ketone to alcohol reduction | c_9.7.92 | Oxo to hydroxyimino |
| c_8.2.2 | Sulfanyl to sulfonyl | c_9.7.57 | Cyano to carbamoyl |
| c_6.2.3 | CO2H—tBu deprotection | c_3.7.2 | Bromo Grignard reaction |
| c_1.1.3 | Iodo N-methylation | c_4.2.2 | 1,2,4-Oxadiazole synthesis |
| c_1.7.6 | Methyl esterification | c_9.1.5 | Hydroxy to bromo |
| c_1.3.1 | Bromo Buchwald-Hartwig amination | c_8.1.5 | Alcohol to ketone oxidation |
| c_6.1.3 | N—Cbz deprotection | c_4.3.3 | Thiazole synthesis |
| c_10.2.1 | Nitration | c_1.2.2 | Aldehyde reductive imination |
| c_1.2.9 | Alcohol + amine condensation | c_1.3.9 | Iodo N-arylation |
| c_9.3.1 | Carboxylic acid to acid chloride | c_3.4.3 | Bromo Stille reaction |
| c_1.7.4 | Hydroxy to methoxy | c_3.1.7 | Iodo Suzuki-type coupling |
| c_2.6.1 | Ester Schotten-Baumann | c_9.7.128 | Carboxy ester to carbamoyl |
| c_9.7.20 | Bromo Miyaura boration | c_6.1.4 | N—Ac deprotection |
| c_3.1.3 | Iodo Suzuki coupling | c_6.1.7 | N—Phth deprotection |
| c_10.1.2 | Chlorination | c_9.7.25 | Bromo to cyano |
| c_8.1.4 | Alcohol to aldehyde oxidation | c_1.2.6 | Ketone reductive imination |
| c_9.7.60 | Deoxygenation | c_4.1.16 | Phillips benzimidazole condensation |
| c_6.1.18 | N-SEM deprotection | c_2.3.7 | CDI urea synthesis |
| c_9.7.112 | Pyridone to chloropyridine | c_11.1 | Chiral separation |
| c_1.2.10 | Formaldehyde reductive amination | c_6.3.9 | O-THP deprotection |
| c_2.6.8 | O-Acetylation | c_8.1.3 | Ketone Dess-Martin oxidation |
| c_2.1.9 | Weinreb amide synthesis | c_2.1.15 | Carboxylic ester + hydrazine reaction |
| c_4.1.24 | Tetrazole synthesis | c_1.7.12 | Alkene ether synthesis |
| | Carboxylic acid + hydrazine | c_6.1.16 | N-Tosyl deprotection |
| c_2.1.3 | condensation | c_1.3.12 | Mesyl N-arylation |
| c_9.7.39 | Chloro to amino | c_6.5.1 | Alkyne TMS deprotection |
| c_7.4.2 | Bouveault-Blanc reduction | c_4.1.59 | Pyrazolamine synthesis |
| c_3.10.1 | Friedel-Crafts acylation | c_3.2.1 | Bromo Heck reaction |
| c_9.7.13 | Azido to amino | c_6.1.11 | N-PMB deprotection |
| c_9.5.4 | Alcohol elimination | c_3.9.13 | Weinreb ketone synthesis |
| c_8.4.2 | Nitrogen oxidation | c_7.7.1 | Alkyne to alkane hydrogenation |
| | Carboxylic acid + sulfonamide | c_10.3.2 | Chlorosulfonation |
| c_2.1.5 | condensation | c_7.9.4 | Ketone to alkane reduction |
| c_3.11.5 | Horner-Wadsworth-Emmons reaction | c_9.7.127 | Krapcho decarboxylation |
| c_9.7.59 | Dechlorination | c_4.2.8 | Prilezhaev epoxidation |
| c_1.7.3 | Ethyl esterification | c_9.1.1 | Appel bromination |
| c_9.7.93 | Oxo to thioxo | c_4.1.4 | Azide-alkyne Huisgen cycloaddition |
| | Isothiocyanate + amine thiourea | c_11.6 | Purification |
| c_2.3.2 | coupling | c_1.1.5 | Bromo Menshutkin reaction |
| c_9.1.7 | Hydroxy to fluoro | c_9.7.139 | Debromination |
| c_4.1.12 | Imidazole synthesis | c_3.11.16 | Wurtz-Fittig coupling |
| c_9.7.61 | Ester hydrolysis | c_3.5.3 | Negishi coupling |
| c_9.7.32 | Bromo to pinacolatoboranyl | c_4.1.2 | 2,5-Pyrroledione synthesis |
| c_3.11.14 | Vilsmeier-Haack reaction | c_3.9.12 | Olefin metathesis |
| c_1.6.12 | Tosyloxy N-alkylation | c_9.7.41 | Chloro to cyano |
| c_9.7.234 | Oxo to difluoro | c_6.1.9 | N-THP deprotection |
| c_9.7.259 | Cyano to Hydroxyamidino | c_8.8.11 | Hydroxylation |
| c_10.4.1 | Formylation | c_3.11.2 | Aldol condensation |
| c_3.1.4 | Triflyloxy Suzuki coupling | c_1.3.5 | Chan-Lam arylamine coupling |
| c_11.7 | Racemization | c_8.5.4 | Ethenyl to formyl |
| c_4.2.3 | 1,3,4-Oxadiazole synthesis | c_10.4.9 | Alkene dihydroxylation |
| c_5.1.5 | N-TFA protection | c_9.7.73 | Hydroxy to azido |
| c_2.1.6 | Carboxylic anhydride + amine reaction | c_10.4.6 | Carboxylation |
| c_2.4.1 | Carbamate Curtius reaction | c_3.9.27 | Lithium Bouveault aldehyde synthesis |
| c_6.1.8 | N—TFA deprotection | c_10.4.3 | Alkene hydration |
| c_9.5.1 | Carbamoyl to cyano | c_9.7.10 | Amino to iodo |
| c_6.5.3 | Ketone dioxolane deprotection | c_4.1.62 | Pyrimidone synthesis |
| c_8.1.2 | Aldehyde Dess-Martin oxidation | c_9.7.102 | Staudinger reduction |
| c_2.6.9 | Steglich esterification | c_11.5 | Isomerization |
| c_3.11.13 | Ullmann-type biaryl coupling | c_1.7.18 | Pinner reaction |
| c_10.1.3 | Fluorination | c_9.7.182 | Cyano to formyl |
| c_9.7.24 | Bromo to borono | c_9.7.194 | Decarboxylation |
| c_4.2.1 | 1,2,4-Oxadiazol-5-one synthesis | c_1.3.10 | Triflyloxy N-arylation |
| c_9.7.3 | Amino to bromo | c_9.7.9 | Amino to hydrazino |
| c_9.7.12 | Amino to isothiocyanato | c_9.7.303 | Mesyloxy to azido |
| c_6.3.16 | O-acetonide deprotection | c_10.4.11 | Upjohn dihydroxylation |
| c_9.7.173 | Amino to hydroxy | c_3.9.14 | Weinreb bromo coupling |
| c_6.5.9 | Aldehyde acetal deprotection | c_3.3.3 | Chloro Sonogashira coupling |
| c_1.8.14 | Fluoro thioether synthesis | c_8.8.3 | Aldehyde to acid oxidation |
| c_10.4.5 | Amination | c_4.2.16 | Oxazole synthesis |
| c_4.1.60 | Pyrazole synthesis | c_9.7.286 | Hydroxyimino to amino |
| c_5.2.1 | CO2H—tBu protection | c_3.7.3 | Chloro Grignard reaction |
| c_3.4.4 | Chloro Stille reaction | c_4.2.4 | Isoxazole synthesis |
| c_9.7.95 | Rosenmund von Braun cyanation | c_9.7.42 | Chloro to fluoro |
| c_1.7.17 | Epoxide + alcohol coupling | c_1.2.3 | Alkylimino-de-oxo-bisubsitution |
| c_1.1.7 | Iodo Menshutkin reaction | c_9.7.147 | Deamination |
| c_2.5.7 | Rathke guanidine synthesis | c_9.7.27 | Bromo to hydroxy |
| c_1.8.6 | S-methylation | c_1.8.15 | Iodo thioether synthesis |
| c_4.1.8 | Knorr pyrazole synthesis | c_5.5.2 | Ketone dioxolane protection |
| c_9.7.4 | Amino to chloro | c_6.1.17 | N—tBu deprotection |
| c_1.7.8 | Ullmann condensation | c_2.1.11 | Hydrazide Schotten-Baumann |
| c_4.1.13 | Indazole synthesis | c_8.8.1 | Methyl to formyl |
| c_2.4.2 | Isocyanate + alcohol reaction | c_4.1.40 | 4-Quinazolinone synthesis |
| c_10.3.1 | Sulfonation | c_8.3.1 | Alcohol to acid oxidation |
| c_6.3.8 | O-MOM deprotection | c_4.2.20 | Dioxolane synthesis |
| c_3.11.6 | Mannich reaction | c_9.7.46 | Chloro to iodo Finkelstein reaction |
| c_1.3.3 | Iodo Buchwald-Hartwig amination | c_9.7.157 | Chloro Kolbe nitrile synthesis |
| c_3.11.52 | Cyanoalkane alkylation | c_9.1.8 | Hydroxy to iodo |
| c_5.3.6 | O-MOM protection | c_3.5.2 | Kumada coupling |
| c_9.7.43 | Chloro to hydrazino | c_4.1.70 | 6-Pyridazinone synthesis |
| c_7.3.5 | Secondary ketimine reduction | c_6.1.2 | N-Bz deprotection |
| c_6.1.20 | N-Besyl deprotection | c_9.7.52 | Chlorosulfonyl to sulfamoyl |
| c_9.7.11 | Amino to isocyanato | c_9.7.82 | Iodo to cyano |
| c_8.8.5 | Lindgren oxidation | c_11.8.3 | Chloride salt formation |
| c_9.7.22 | Bromo to amino | c_9.7.155 | Bromo Kolbe nitrile synthesis |
| c_3.4.5 | Iodo Stille reaction | c_1.7.1 | Chan-Lam ether coupling |
| c_7.9.12 | Pyridine to piperidine hydrogenation | c_1.8.7 | Migita thioether synthesis |
| c_6.1.6 | N—Fmoc deprotection | c_6.5.2 | Aldehyde dioxolane deprotection |
| c_3.11.31 | Henry reaction | c_9.7.183 | Hydroxy to amino |
| c_2.3.6 | Urea Curtius reaction | c_4.2.17 | 1,3-Benzoxazole synthesis |
| c_2.1.18 | Formic acid + amine condensation | c_7.3.4 | Secondary aldimine reduction |
| c_9.7.177 | Bromo to carboxy | c_9.7.164 | Carboxylic acid Schmidt reaction |
| c_4.1.48 | Pyrimidine synthesis | c_3.11.1 | Aldol addition |
| c_9.7.74 | Hydroxy to mesyloxy | c_1.9.12 | Phosphorus Menshutkin reaction |
| c_9.7.23 | Bromo to azido | c_2.3.4 | Amino to ureido |
| c_9.7.40 | Chloro to azido | c_9.7.44 | Chloro to hydroxy |
| c_9.7.264 | Hydroxy to difluoromethoxy | c_8.1.9 | Aldehyde Collins oxidation |
| c_9.7.181 | Cyano to amidino | c_3.11.11 | Strecker ketone reaction |
| c_1.6.11 | Triflyloxy N-alkylation | c_4.1.53 | 1,2,4-Triazole synthesis |
| c_3.1.8 | Triflyloxy Suzuki-type coupling | c_4.2.39 | 1,3-Dioxane synthesis |
| c_6.4.1 | S-carbonyl deprotection | c_9.7.140 | Defluorination |
| c_3.9.2 | Bromo ketone Barbier reaction | c_9.7.148 | Imine hydrolysis |
| c_5.1.2 | N—Cbz protection | | Aldehyde Ruppert-Prakash |
| c_1.2.4 | Eschweiler-Clarke methylation | c_3.11.81 | trifluoromethylation |
| c_3.5.1 | Hiyama coupling | c_7.9.8 | Alkyne to alkene hydrogenation |
| c_9.3.3 | Sulfo to chlorosulfonyl | c_9.7.141 | Deiodination |
| c_3.7.14 | Bromo Grignard + ester reaction | c_2.8.5 | S-Thioester synthesis |
| c_4.1.56 | Isoindolinone synthesis | c_5.5.1 | Aldehyde dioxolane protection |
| c_3.9.1 | Bromo aldehyde Barbier reaction | c_ 3.2.3 | Iodo Heck reaction |
| c_4.3.9 | Benzothiazole synthesis | c_9.7.64 | Fluoro to amino |
| c_1.7.13 | Ether synthesis | | Debus-Radziszewski imidazole |
| c_6.1.13 | N-Benzhydrylidene deprotection | c_4.1.89 | synthesis |
| c_11.2 | Dehydration | c_9.7.103 | Triflyloxy Miyaura boration |
| c_4.1.61 | Pyrazolone synthesis | c_8.2.4 | Sulfinyl to sulfonyl |
| c_2.5.5 | Nitrile + amine reaction | c_2.1.34 | Amino to formamido |
| c_4.1.14 | Paal-Knorr pyrrole synthesis | c_9.7.8 | Amino to guanidino |
| c_5.1.3 | N-Fmoc protection | c_9.7.165 | Chlorocarbonyl to carbamoyl |
| c_3.9.41 | Decarboxylative coupling | c_5.3.1 | O—Bn protection |
| | [1,2,4] Triazolo [4,3-a] pyridine | c_4.3.8 | 1,3,4-Thiadiazole synthesis |
| c_4.1.69 | synthesis | c_9.7.30 | Bromo to mesyl |
| c_3.10.2 | Friedel-Crafts alkylation | c_8.5.2 | Ozonolysis |
| c_4.3.11 | Thiazoline synthesis | c_9.7.85 | Iodo to pinacolatoboranyl |
| c_6.3.3 | O-TIPS deprotection | c_2.1.27 | Imidazolecarbonyl to amide |
| c_1.9.1 | Michaelis-Arbuzov reaction | c_3.11.69 | Aromatic Claisen rearrangement |
| c_9.7.170 | Amino to chlorosulfonyl | c_5.3.4 | O-TMS protection |
| c_10.4.17 | Pinacolatoborylation | c_4.1.3 | 2-Pyrrolidone synthesis |
| c_9.7.166 | Formyl to cyano | c_4.1.35 | Borsche-Drechsel carbazole synthesis |
| c_9.7.58 | Cyano to thiocarbamoyl | c_1.3.4 | Triflyloxy Buchwald-Hartwig amination |
| c_4.1.22 | Pyrroldine synthesis | c_9.7.7 | Amino to fluoro |
| c_4.3.6 | Benzothiophene synthesis | c_5.1.7 | N-Bz protection |
| c_1.1.6 | Chloro Menshutkin reaction | c_4.1.20 | Piperazine synthesis |
| c_4.1.55 | Dihydropyridine synthesis | c_9.7.37 | Chloro Miyaura boration |
| c_6.1.12 | N-Benzhydryl deprotection | c_8.4.1 | Amino to nitro |
| c_6.3.10 | O-PMB deprotection | c_1.9.8 | Stannylation |
| c_8.3.2 | Jones acid oxidation | | Ketone Ruppert-Prakash |
| | Bromo Grignard + nitrile ketone | c_3.11.82 | trifluoromethylation |
| c_3.7.10 | synthesis | c_9.7.34 | Carboxy to carbonazidoyl |
| c_9.7.265 | Formamido to amino | c_9.7.16 | Borono to hydroxy |
| c_5.3.3 | O-TIPS protection | c_9.7.236 | Mesyloxy Kolbe nitrile synthesis |
| c_4.1.49 | Pyridine synthesis | c_3.9.5 | Iodo aldehyde Barbier reaction |
| c_10.4.16 | Borylation | c_9.7.78 | Iodo Miyaura boration |
| c_9.7.174 | Amino to mesylamino | c_4.1.42 | Indole synthesis |
| | Johnson-Corey-Chaykovsky | c_2.6.10 | Bromo alkoxycarbonylation |
| c_4.2.9 | epoxidation | c_9.7.267 | Bromo to chloro |
| c_10.4.15 | Nitrosylation | c_4.3.4 | Gewald reaction |
| c_8.1.10 | Ketone Collins oxidation | c_9.7.70 | Hofmann reaction |
| c_1.1.2 | Menshutkin reaction | c_4.1.11 | Larock indole synthesis |
| c_7.9.6 | Wolff-Kishner reduction | c_3.11.10 | Strecker aldehyde reaction |
| c_7.5.2 | Corey-Itsuno reduction | c_9.7.248 | Triflyloxy to cyano |
| c_9.7.29 | Bromo to iodo Finkelstein reaction | c_4.2.29 | Van Leusen oxazole synthesis |
| c_9.7.136 | Chloro to sulfanyl | c_8.1.7 | Ketone Jones oxidation |
| c_4.1.93 | Biginelli reaction | c_6.3.4 | O-TMS deprotection |
| c_2.5.2 | Imidic ester + amine reaction | c_9.7.5 | Amino to cyano |
| c_1.9.4 | Bromo stannylation | c_4.1.46 | Benzimidazolone synthesis |
| c_4.2.35 | Oxirane synthesis | c_8.7.4 | Alkene oxidation |
| c_9.7.94 | Pinacolatoboranyl to borono | c_4.2.5 | Morpholine synthesis |
| c_3.9.23 | Simmons-Smith reaction | c_4.2.22 | Benzofuran synthesis |
| c_8.5.1 | Alkene oxidative cleavage | c_4.1.101 | Carbazole synthesis |
| c_3.11.3 | Diels-Alder cycloaddition | c_2.3.5 | Amino to thioureido |
| c_4.1.34 | Fischer indole synthesis | c_4.2.18 | 3,1-Benzoxazin-4-one synthesis |
| c_9.7.179 | Chloro to bromo | c_2.6.4 | Baeyer-Villiger oxidation |
| c_4.1.71 | Quinazoline synthesis | c_3.9.24 | Nickel Kumada coupling |
| c_1.6.1 | Bromo Gabriel alkylation | c_3.11.39 | Alkyne + ketone reaction |
| c_9.7.306 | Iodo to trifluoromethyl | c_6.5.10 | Ketone ketal deprotection |
| c_3.3.5 | Triflyloxy Sonogashira coupling | c_4.2.32 | 2-Oxazoline synthesis |
| c_8.1.25 | Aldehyde Ley-Griffith oxidation | c_9.7.54 | Corey-Fuchs reaction step 1 |
| c_3.11.44 | Houben-Hoesch reaction | c_5.1.4 | N—Phth protection |
| c_3.9.60 | Negishi-type coupling | c_7.9.19 | Aldehyde to alkane reduction |
| c_3.2.5 | Bromo Heck-type reaction | c_1.8.1 | Sulfinic acid + bromide reaction |
| c_9.7.2 | Amino to azido | c_9.7.224 | Oxo to cyano |
| c_4.1.57 | Lactam synthesis | c_3.5.4 | Palladium Kumada coupling |
| | Johnson-Corey-Chaykovsky | c_7.9.5 | Nitroso to amino reduction |
| c_3.11.27 | cyclopropane synthesis | | Carboxylic anhydride + hydrazine |
| c_2.1.8 | Thioamide Schotten-Baumann | c_2.1.12 | reaction |
| c_9.5.5 | Hydroxyiminomethyl to cyano | | [1,2,4] Triazolo [1,5-a] pyridine |
| c_11.3 | Hydration | c_4.1.90 | synthesis |
| c_1.7.2 | Diazomethane esterification | c_9.7.81 | Iodo to borono |
| c_9.7.232 | Fluoro to chloro | | Seyferth-Gilbert-Bestmann aldehyde |
| c_2.1.24 | Ketone Schmidt reaction | c_9.7.100 | reaction |
| c_8.1.18 | Periodate cleavage | c_4.3.10 | 1,3-Benzothiazin-4-one synthesis |
| c_9.7.28 | Bromo to iodo | c_9.7.99 | Sandmeyer iodination |
| c_4.2.21 | Chromanone synthesis | c_9.7.178 | Bromo to fluoro |
| c_3.9.55 | Rieche formylation | c_3.9.44 | Minisci reaction |
| c_4.1.5 | Bischler-Napieralski reaction | c_3.9.17 | Weinreb iodo coupling |
| c_3.4.6 | Triflyloxy Stille reaction | c_9.1.2 | Appel chlorination |
| c_1.7.14 | O-methylation | c_1.8.10 | Epoxide + thiol coupling |
| c_9.7.45 | Chloro to iodo | c_4.2.11 | 1,2-Benzoxazole synthesis |
| c_4.1.58 | Pyazine synthesis | c_8.4.3 | Tertiary amine oxidation |
| c_9.7.253 | Bromo elimination | c_9.7.65 | Fluoro to hydrazino |
| c_3.9.26 | Grignard Bouveault aldehyde synthesis | c_9.7.48 | Chloro to methoxy |
| c_3.11.70 | Indole + ketone condensation | c_4.1.68 | 2,4-Quinazolinedione synthesis |
| c_9.7.66 | Fluoro to hydroxy | c_4.1.32 | Pfitzinger reaction |
| c_1.6.3 | Chloro Gabriel alkylation | c_4.1.47 | Benzotriazole synthesis |
| c_9.7.261 | Cyanohydrin reaction | c_2.5.4 | Thioimidic ester + amine reaction |
| c_4.1.117 | Bucherer-Bergs reaction | c_9.7.260 | Boekelheide reaction |
| c_8.1.26 | Ketone Ley-Griffith oxidation | c_4.1.6 | Cyclic Beckmann rearrangement |
| c_2.1.25 | Amino to imidazolecarboxamido | c_1.1.8 | Triflyloxy Menshutkin reaction |
| c_9.7.245 | Chlorocarbonyl to carboxy | c_3.9.67 | Kulinkovich-Szymoniak reaction |
| c_10.1.6 | Alkene bromination | c_1.9.11 | Hirao coupling |
| c_4.1.100 | Pyrrole synthesis | c_2.1.26 | Carboxy to imidazolecarbonyl |
| c_3.9.28 | McMurry coupling | | N-Dimethylaminomethylene |
| c_3.9.6 | Iodo ketone Barbier reaction | c_6.1.15 | deprotection |
| c_9.7.135 | Bromo to sulfanyl | c_3.11.24 | Ortho Fries rearrangement |
| c_3.2.7 | Iodo Heck-type reaction | c_8.8.6 | Pinnick oxidation |
| c_1.9.7 | Iodo stannylation | c_3.8.2 | Wittig-type olefination |
| c_9.7.212 | Mesyloxy to iodo | c_3.9.25 | Bouveault aldehyde synthesis |
| c_11.8.1 | Acetate salt formation | c_1.1.1 | Chan-Lam alkylamine coupling |
| c_9.7.206 | Iodo to carboxy | c_4.2.28 | 1,3,4-Oxadiazol-2-one synthesis |
| c_9.7.268 | Carboxy to cyano | c_8.1.28 | Aldehyde Parikh-Doering oxidation |
| c_9.7.19 | Bromo Grignard preparation | c_9.7.67 | Fluoro to methoxy |
| c_6.3.5 | Silyl ether deprotection | c_5.1.6 | N-Bn protection |
| c_4.1.23 | Skraup reaction | c_8.1.14 | Aldehyde Cornforth oxidation |
| c_2.1.37 | Carboxylic acid + imine condensation | c_3.11.42 | Dieckmann condensation |
| c_4.1.21 | Piperidine synthesis | c_9.7.300 | Isothiocyanato to thioureido |
| c_4.2.40 | 1,4-Dioxane synthesis | c_8.8.2 | Delepine aldehyde oxidation |
| c_4.1.72 | Pyrazolo [1,5-a] pyrimidine synthesis | c_4.1.30 | Niementowski quinazoline synthesis |
| c_9.7.254 | Chloro elimination | | Chloro Grignard + nitrile ketone |
| c_3.11.38 | Alkyne + aldehyde reaction | c_3.7.11 | synthesis |
| c_3.11.17 | Wurtz-type coupling | c_7.9.14 | Disulfide reduction |
| c_9.7.163 | Fluoro to cyano | c_9.7.107 | Iodo to azido |
| c_8.1.1 | Bromo to oxo oxidation | c_9.7.104 | Triflyloxy to pinacolatoboranyl |
| c_9.7.142 | Bromo to formyl | c_9.7.249 | Bromo to chlorosulfonyl |
| c_2.1.30 | Bromo aminocarbonylation | c_3.7.13 | Iodo Grignard + nitrile ketone synthesis |
| c_5.3.7 | O-acetonide protection | | Carboxylic anhydride + sulfonamide |
| c_4.2.27 | 1,3-Benzoxazol-2-one synthesis | c_2.1.14 | reaction |
| c_8.1.15 | Ketone Cornforth oxidation | c_9.7.238 | Tosyloxy Kolbe nitrile synthesis |
| c_1.8.16 | Acetoxy thioether synthesis | c_2.2.4 | Sulfonic acid + amine reaction |
| c_9.7.305 | Bromo to trifluoromethyl | c_8.8.14 | Riley oxidation |
| c_7.5.5 | Luche reduction | c_9.7.207 | Decarbonylation |
| c_9.7.190 | Carboxy to bromo | c_4.1.27 | Conrad-Limpach quinoline synthesis |
| c_2.8.4 | Carboxylic acid + thiol condensation | c_9.7.134 | Sulfanyl to chlorosulfonyl |
| c_9.7.262 | Urech cyanohydrin method | c_9.7.285 | Hydroxyamidino to amidino |
| c_3.7.5 | Iodo Grignard reaction | c_11.8.4 | Mesylate salt formation |
| c_9.7.109 | Chlorosulfonyl to sulfanyl | c_9.7.69 | Formyl to ethynyl |
| c_9.7.226 | Chloro to isothiocyanato | c_1.7.16 | Aziridine + alcohol coupling |
| c_9.7.6 | Amino to diazonio | c_9.7.241 | Bromoform reaction |
| c_9.7.90 | Newman-Kwart rearrangement | c_9.7.289 | Tosyloxy to fluoro |
| c_4.2.45 | Morpholin-3-one synthesis | c_8.8.16 | Phosphorus oxidation |
| c_9.7.47 | Chloro to mesyl | c_9.7.68 | Formamido to isocyano |
| c_3.9.37 | Reformatsky reaction | c_4.1.7 | Doebner-Miller reaction |
| c_9.7.209 | Mesyloxy to bromo | c_4.1.86 | Imidazo [1,2-a] pyridine synthesis |
| c_3.11.35 | Perkin condensation | c_2.8.3 | Phosphonamide Schotten-Baumann |
| c_9.7.223 | Van Leusen reaction | c_6.3.11 | O-Tosyl deprotection |
| c_6.4.2 | S-thiocarbamate deprotection | c_9.7.184 | Mesyl to cyano |
| c_9.7.204 | Chloro to carboxy | c_9.7.87 | Mesyloxy to hydroxy |
| c_9.7.31 | Bromo to methoxy | c_5.5.7 | Aldehyde dithiane protection |
| c_9.7.110 | Chlorosulfonyl to sulfinato | c_2.6.11 | Chloro alkoxycarbonylation |
| c_2.6.7 | Imidazolecarbonyl to ester | c_2.1.17 | Carboxylic ester + sulfonamide reaction |
| c_9.3.2 | Acid to acid chloride | c_9.7.106 | Fluoro to azido |
| c_1.8.4 | Sulfinic acid + iodide reaction | c_9.7.186 | Nitro to hydroxyamino |
| c_3.11.55 | Blanc chloromethylation | c_1.3.14 | Tosyloxy N-arylation |
| c_1.2.13 | Aziridine + amine coupling | c_9.7.21 | Bromo Negishi preparation |
| c_7.9.11 | Nitrosamine to hydrazine reduction | c_4.1.9 | Knorr quinoline cyclization |
| c_9.7.230 | Regitz diazo transfer | c_4.1.66 | Wenker synthesis |
| c_4.1.1 | 1,2,3-Triazole synthesis | c_4.1.94 | Benzimidazolethione synthesis |
| c_9.7.217 | Pummerer rearrangement | c_9.7.150 | Hydroxyimino to oxo |
| c_2.6.6 | Hydroxy to imidazolecarbonyloxy | c_3.9.47 | Julia-Kocienski olefination |
| c_3.9.4 | Chloro ketone Barbier reaction | c_9.7.302 | Garigipati amidine synthesis |
| c_1.2.8 | Ketone Leuckart reaction | c_2.6.13 | Iodo alkoxycarbonylation |
| c_4.2.26 | 3,1-Benzoxazine-2,4-dione synthesis | c_8.1.23 | Aldehyde Swern oxidation |
| c_9.7.26 | Bromo to hydrazino | c_1.2.12 | Dimethyl acetal reductive amination |
| c_7.1.2 | Zinin reaction | c_5.5.3 | Aldehyde dioxane protection |
| c_3.9.54 | Tebbe olefination | c_1.6.6 | Fluoro N-alkylation |
| c_4.1.19 | Pinner pyrimidine synthesis | c_9.7.266 | Hydroxy to tosyloxy |
| c_3.2.2 | Chloro Heck reaction | c_9.7.307 | von Braun reaction |
| c_1.9.3 | Hydrostannylation | c_6.5.8 | Ketone dithiane deprotection |
| c_5.5.6 | Ketone dithiolane protection | c_1.8.3 | Sulfinic acid + fluoride reaction |
| c_9.7.83 | Iodo to hydroxy | c_9.7.258 | Mesyl to methoxy |
| c_2.1.28 | Ugi reaction | c_6.3.13 | O-SEM deprotection |
| c_9.7.116 | Diazo to bromo | c_9.7.227 | Iodo to mesyl |
| c_1.8.2 | Sulfinic acid + chloride reaction | c_9.7.291 | Thioxo to imino |
| c_4.1.111 | Pyrimidine-2,4,6-trione synthesis | c_3.11.46 | Baylis-Hillman reaction |
| c_9.7.55 | Corey-Fuchs reaction step 2 | c_4.1.29 | Friedlander quinoline synthesis |
| c_8.5.3 | Lemieux-Johnson oxidation | c_8.8.7 | Bromo Kornblum oxidation |
| c_9.7.17 | Borono to pinacolatoboranyl | c_3.7.17 | Iodo Grignard + ester reaction |
| c_9.7.75 | Hydroxy to sulfanyl | c_4.3.18 | Thiazol-2-imine synthesis |
| c_4.1.63 | Pyridone synthesis | c_3.11.78 | Stetter reaction |
| c_9.7.188 | Thiocyanato to sulfanyl | c_4.1.67 | 2,3-Quinoxalinedione synthesis |
| c_3.11.76 | Michael-Henry reaction | c_8.1.13 | Ketone Sarett oxidation |
| c_2.5.6 | Nitrile + hydrazine reaction | c_6.5.11 | Aldehyde dioxane deprotection |
| c_3.7.15 | Chloro Grignard + ester reaction | c_3.2.4 | Triflyloxy Heck reaction |
| c_9.7.49 | Chloro to pinacolatoboranyl | c_9.7.299 | Isocyanato to ureido |
| c_11.8.5 | Trifluoroacetate salt formation | c_2.3.3 | Levy reaction |
| c_9.7.231 | Fluoro to bromo | c_9.7.130 | Chlorosulfonyl to sulfo |
| c_4.1.108 | Hemetsberger indole synthesis | c_3.11.32 | Knunyants fluoroalkylation |
| | Kishner diazomethane cyclopropane | c_9.7.219 | Bromo to thiocyanato |
| c_3.11.57 | synthesis | c_3.9.3 | Chloro aldehyde Barbier reaction |
| c_4.2.19 | Dihydroisoxazole synthesis | c_9.7.290 | Tosyloxy to iodo |
| c_3.9.68 | Aldehyde Corey-Seebach reaction | c_1.3.11 | Chichibabin amination |
| c_9.7.252 | Desulfonylation | c_3.11.12 | Triple bond Diels-Alder |
| c_9.7.284 | Mesyl to hydroxy | c_9.7.185 | Nitro to fluoro |
| c_4.1.85 | Pyrazolo [1,5-a] pyridine synthesis | c_10.1.9 | Alkene hydrochlorination |
| c_10.1.8 | Alkene hydrobromination | c_8.1.31 | Glycol cleavage |
| c_9.7.225 | Fluoro to sulfanyl | c_9.7.111 | Chlorosulfonyl to sulfino |
| c_1.9.5 | Chloro stannylation | c_2.1.33 | Iodo aminocarbonylation |
| c_4.1.75 | Phthalazinone synthesis | c_3.9.18 | Cadiot-Chodkiewicz coupling |
| c_9.7.36 | Chloro Grignard preparation | c_10.1.7 | Alkene chlorination |
| c_3.2.6 | Chloro Heck-type reaction | c_4.1.76 | Pyridotriazole synthesis |
| c_11.8.2 | Bromide salt formation | c_8.7.1 | Rubottom oxidation |
| c_9.7.96 | Sandmeyer bromination | c_7.9.15 | Birch reduction |
| c_8.2.3 | Sulfinimidoyl to sulfonimidoyl | c_4.1.107 | Pyridine N-oxide rearrangement |
| c_3.9.65 | Kulinkovich reaction | c_8.7.3 | Ethenyl to acetyl |
| c_1.8.8 | Disulfide coupling | c_9.7.187 | Pinacolatoboranyl to bromo |
| | Ciamician-Dennstedt cyclopropane | c_9.7.304 | Triflyloxy to azido |
| c_3.11.80 | synthesis | c_4.1.10 | Knorr quinoline synthesis |
| c_4.2.48 | 1,3-Oxazine-2,4-dione synthesis | c_7.5.4 | Meerwein-Ponndorf-Verley reduction |
| c_9.7.86 | Isocyanato to amino | c_8.1.12 | Aldehyde Sarett oxidation |
| c_3.11.54 | Ketene S, S-acetal synthesis | c_8.6.1 | Riley hydroxylation |
| c_9.7.242 | Chloroform reaction | c_7.9.20 | Phosphoryl deoxygenation |
| c_4.1.83 | Quinoxalinone synthesis | c_4.2.42 | Sharpless epoxidation |
| c_11.8.9 | Sulfate salt formation | c_9.7.287 | Tosyloxy to bromo |
| c_4.2.37 | Pechmann condensation | c_2.1.21 | Alcohol Ritter reaction |
| c_4.1.98 | Van Leusen imidazole synthesis | c_9.7.71 | Hydrazino to amino |
| c_9.7.211 | Mesyloxy to fluoro | c_6.1.10 | N-PMP deprotection |
| c_4.1.103 | Borsche-Drechsel cyclization | c_3.11.36 | Perkin reaction |
| c_5.5.4 | Ketone dioxane protection | c_9.7.88 | Methylsulfanyl to hydrazino |
| c_9.7.89 | Nef reaction | c_7.9.13 | Pyrazine to piperazine hydrogenation |
| c_4.1.54 | 2,4-Pyrimidinedione synthesis | c_4.2.23 | Oxa-Diels-Alder reaction |
| c_2.1.38 | Carboxylic anhydride + imine reaction | c_4.3.12 | Dithiane Gewald reaction |
| c_9.7.80 | Iodo to amino | c_4.1.118 | 2-Quinazolinone synthesis |
| c_9.7.240 | Acetyl to carboxy | c_4.2.47 | 1,3-Benzoxazine-2,4-dione synthesis |
| c_4.2.7 | Oxa Pictet-Spengler reaction | c_2.1.35 | Nitro to formamido |
| c_9.7.51 | Chlorosulfonyl to fluorosulfonyl | c_2.8.6 | O-Thioester synthesis |
| c_9.7.220 | Chloro to thiocyanato | c_8.7.2 | Wacker-Tsuji oxidation |
| c_9.7.145 | Iodo to formyl | c_3.11.51 | Petasis reaction |
| c_9.7.138 | Amino to sulfanyl | c_4.3.7 | Isothiazole synthesis |
| c_3.11.73 | Ketone Peterson olefination | c_9.7.161 | Iodo Kolbe nitrile synthesis |
| c_3.11.25 | Para Fries rearrangement | c_2.1.36 | Willgerodt-Kindler reaction |
| c_9.7.255 | Iodo elimination | c_8.1.29 | Ketone Parikh-Doering oxidation |
| c_6.1.14 | N-Benzylidene deprotection | c_10.4.10 | Milas hydroxylation |
| c_3.9.34 | Hydroformylation | c_3.11.60 | Chloro Nierenstein reaction |
| c_9.7.56 | Curtius rearrangement | c_3.11.45 | Kolbe-Schmitt reaction |
| c_2.4.3 | Isothiocyanate + alcohol reaction | c_10.4.8 | Reimer-Tiemann formylation |
| c_1.9.10 | Abramov reaction | c_10.4.12 | Sharpless asymmetric dihydroxylation |
| c_2.2.1 | Sulfinamide Schotten-Baumann | c_2.6.5 | Yamaguchi esterification |
| c_9.7.293 | Carboxylic acid to peroxyacid | c_4.1.18 | Pictet-Spengler reaction |
| c_2.1.39 | Carboxylic ester + imine reaction | c_3.11.56 | Blanc bromomethylation |
| c_3.9.61 | Blaise ketone synthesis | c_2.8.1 | Acyclic Beckmann rearrangement |
| c_9.7.208 | Cyano to hydroxy | c_9.7.113 | Wolff rearrangement |
| c_11.8.8 | Sodium salt formation | c_4.2.6 | Paal-Knorr furan synthesis |
| c_5.5.8 | Ketone dithiane protection | c_4.1.74 | Benzimidazolimine synthesis |
| c_8.1.24 | Ketone Swern oxidation | c_6.1.19 | N-Bus deprotection |
| c_9.7.257 | Curtius degradation | c_4.1.65 | Pomeranz-Fritsch reaction |
| c_1.6.7 | Iodo Gabriel alkylation | c_8.1.30 | Criegee oxidation |
| c_3.9.29 | Bromo formaldehyde Barbier reaction | c_4.2.10 | Gewald furan synthesis |
| c_3.9.66 | Kulinkovich-de Meijere reaction | c_3.9.74 | Ketone Corey-Seebach reaction |
| c_9.7.84 | Iodo to methylsulfanyl | c_8.1.21 | Chloro to oxo oxidation |
| c_1.2.11 | Formaldehyde reductive imination | c_4.3.5 | Thiophene synthesis |
| c_1.8.11 | Leuckart thiophenol reaction | c_9.7.229 | Disulfide to chlorosulfonyl |
| c_1.9.13 | Triflyloxy stannylation | c_9.7.200 | Bromo Hunsdiecker reaction |
| c_4.3.19 | Thiirane synthesis | c_9.7.250 | Chloro to chlorosulfonyl |
| c_4.1.80 | Aza-Diels-Alder reaction | c_4.1.77 | Phthalazine synthesis |
| c_6.5.7 | Aldehyde dithiane deprotection | c_6.5.4 | Ketone dioxane deprotection |
| c_9.7.133 | Mesyloxy to cyano | c_9.7.175 | Amino to thiocyanato |
| c_4.1.95 | 2-Thioxopyrimidin-4-one synthesis | c_9.7.251 | Iodo to chlorosulfonyl |
| c_3.11.7 | Robinson annulation | c_9.7.137 | Iodo to sulfanyl |
| c_9.7.79 | Iodo Negishi preparation | c_3.9.69 | Bromo Corey-Seebach reaction |
| c_9.7.180 | Chloro to nitro | c_9.7.115 | Diazo to chloro |
| c_4.1.114 | 1,2,4-Triazol-3-one synthesis | c_4.1.36 | Gassman indolone synthesis |
| c_1.3.13 | Mesyloxy N-arylation | c_9.7.167 | Amino to isocyano |
| c_9.7.14 | Balz-Schiemann reaction | c_9.7.18 | Bromo Gabriel synthesis |
| c_3.11.53 | Nitroalkane alkylation | c_9.7.149 | Iminium hydrolysis |
| c_3.4.2 | Stille-Kelly coupling | c_4.1.15 | Pechmann pyrazole synthesis |
| c_4.1.73 | Knorr pyrrole synthesis | c_3.11.74 | Olefin hydroalkylation |
| c_7.9.3 | Clemmensen reduction | c_4.3.2 | Paal-Knorr thiophene synthesis |
| c_10.1.15 | Sulfur chlorination | c_7.9.16 | Fukuyama reduction |
| c_9.7.1 | Amino to ammonio | c_9.7.172 | Chloro to sulfo |
| c_8.1.6 | Aldehyde Jones oxidation | c_3.11.26 | Aldehyde Hosomi-Sakurai reaction |
| c_4.1.99 | 1,2,4-Triazole-3-thione synthesis | c_9.7.35 | Chloro Gabriel synthesis |
| c_3.5.7 | Triflyloxy Hiyama coupling | c_9.7.295 | Carboxylic anhydride to peroxyacid |
| c_2.1.31 | Chloro aminocarbonylation | c_3.9.46 | Modified Julia olefination |
| c_4.1.79 | Azide-nitrile Huisgen cycloaddition | c_3.9.59 | Nickel Negishi coupling |
| c_3.11.63 | Betti reaction | c_4.2.33 | Aldehyde Darzens reaction |
| c_11.8.11 | Iodide salt formation | c_1.8.9 | Aziridine + thiol coupling |
| c_9.7.210 | Mesyloxy to chloro | c_11.8.7 | Potassium salt formation |
| c_11.8 | Salt formation | | [1,2,4] Triazolo [4,3-a] pyridin-3-one |
| c_10.2.4 | Menke nitration | c_4.1.84 | synthesis |
| c_8.8.4 | Fleming-Tamao oxidation | c_3.11.15 | Wurtz coupling |
| c_3.11.37 | Alkyne + formaldehyde reaction | c_9.7.118 | Iodo to hydrazino |
| c_9.7.101 | Seyferth-Gilbert aldehyde reaction | c_4.2.13 | Bromolactonization |
| c_5.5.5 | Aldehyde dithiolane protection | c_3.11.41 | Claisen condensation |
| c_4.1.113 | Pyrimidine-4,6-dione synthesis | c_4.1.26 | Combes quinoline synthesis |
| c_4.1.64 | Bischler-Mohlau indole synthesis | c_9.7.122 | Diazonio to iodo |
| c_3.10.3 | Scholl reaction | c_11.8.10 | Bisulfate salt formation |
| c_8.1.16 | Oppenauer oxidation | c_4.1.116 | Hydantoin synthesis |
| c_6.5.6 | Ketone dithiolane deprotection | c_4.3.14 | 1,2,4-Thiadiazole synthesis |
| c_4.1.31 | Niementowski quinoline synthesis | c_9.7.38 | Chloro Negishi preparation |
| c_3.9.43 | Blaise reaction | c_3.11.23 | Ketone Hosomi-Sakurai reaction |
| c_4.1.28 | Doebner reaction | c_9.7.77 | Iodo Grignard preparation |
| c_9.7.143 | Chloro to formyl | c_3.9.22 | Cadiot-Chodkiewicz-type coupling |
| c_1.2.15 | Japp-Klingemann reaction | c_3.2.8 | Triflyloxy Heck-type reaction |
| c_2.1.29 | Passerini reaction | c_3.11.72 | Aldehyde Peterson olefination |
| c_4.2.12 | Yamaguchi lactonization | c_4.2.34 | Ketone Darzens reaction |
| c_3.9.50 | Iodo Takai olefination | c_9.7.53 | Corey-Fuchs reaction |
| c_9.7.214 | Cope elimination | c_10.1.11 | Alkyne to alkene bromination |
| c_3.9.70 | Chloro Corey-Seebach reaction | c_9.7.256 | Emde degradation |
| c_11.9.3 | Sodium salt separation | c_4.1.52 | Boennemann cyclization |
| c_10.4.14 | Baudisch reaction | c_11.9.1 | Lithium salt separation |
| c_9.7.72 | Hydrazino to bromo | c_4.3.16 | Hinsberg thiophene synthesis |
| c_9.7.105 | Zincke nitration | c_4.1.44 | Hydroquinazolinone synthesis |
| c_9.7.91 | Nitro to hydrazino | c_4.1.119 | Pyrrolidinium synthesis |
| c_7.3.3 | Primary ketimine reduction | | Chlorocarbonyl Tsuji-Wilkinson |
| | [1,2,4] Triazolo [1,5-a] pyrimidin-7-one | c_9.7.276 | decarbonylation |
| c_4.1.97 | synthesis | | Kaiser-Johnson-Middleton dinitrile |
| c_3.11.65 | Oxy-Cope rearrangement | c_4.1.120 | cyclization |
| c_9.7.233 | Fluoro to iodo | c_9.7.288 | Tosyloxy to chloro |
| c_7.9.21 | Crossed Cannizzaro reaction | | 2-Thioxopyrimidine-4,6-dione |
| c_9.7.222 | Iodo to thiocyanato | c_4.1.112 | synthesis |
| c_7.5.3 | Noyori asymmetric hydrogenation | c_10.1.13 | Alkyne to alkene chlorination |
| c_9.7.126 | Sulfoxy to hydroxy | c_2.7.1 | Sulfinic ester Schotten-Baumann |
| c_3.11.75 | Aza-Henry reaction | c_3.9.73 | Iodo Corey-Seebach reaction |
| c_3.11.29 | Favorskii rearrangement | c_3.9.35 | Pauson-Khand reaction |
| c_4.3.17 | 1,2-Dithiolane synthesis | c_4.1.17 | Pictet-Spengler cyclization |
| c_4.1.110 | Nenitzescu indole synthesis | c_3.9.75 | Corey-House synthesis |
| c_3.11.62 | Darzens tetralin synthesis | c_3.11.43 | Hammick reaction |
| c_4.2.44 | 1,3-Oxazinane synthesis | c_8.1.20 | Ketone Corey-Kim oxidation |
| c_4.2.15 | Iodolactonization | c_8.1.27 | Retro-Henry reaction |
| c_9.7.124 | Diazonio to hydroxy | c_9.7.205 | Fluoro to carboxy |
| c_2.1.19 | Ritter reaction | c_9.7.263 | Ultee cyanohydrin method |
| c_3.11.33 | Nazarov cyclization | c_9.7.270 | Stephen aldehyde synthesis |
| c_8.8.8 | Chloro Kornblum oxidation | c_9.7.168 | Hofmann isonitrile synthesis |
| c_3.11.64 | Cope rearrangement | c_3.9.72 | Fluoro Corey-Seebach reaction |
| c_7.9.18 | Ketone Mozingo reduction | c_3.9.63 | Gomberg-Bachmann reaction |
| c_3.9.64 | Meerwein arylation | c_3.11.59 | Bromo Nierenstein reaction |
| c_3.11.22 | Ketal Hosomi-Sakurai reaction | c_4.1.102 | Bucherer carbazole synthesis |
| c_9.7.125 | Hofmann rearrangement | c_3.9.38 | Castro-Stephens coupling |
| c_4.2.43 | Paterno-Buchi reaction | c_3.11.77 | Tiffeneau-Demjanov rearrangement |
| c_4.1.115 | Urech hydantoin synthesis | c_4.2.36 | Jacobsen epoxidation |
| c_3.11.71 | Riessert reaction | c_9.7.228 | Zincke disulfide cleavage |
| c_3.9.31 | Iodo formaldehyde Barbier reaction | c_9.7.237 | Mesyloxy Kolbe isonitrile synthesis |
| c_3.11.68 | Claisen rearrangement | c_9.7.197 | Chloro Kochi reaction |
| c_4.1.109 | Reissert indole synthesis | c_9.7.283 | Diketone decarbonylation |
| c_3.11.50 | Barton-Kellogg olefination | c_9.7.195 | Barton decarboxylation |
| c_9.7.171 | Amino to sulfo | c_9.7.244 | Bromocarbonyl to carboxy |
| c_1.7.10 | Perkow reaction | c_2.8.7 | Atherton-Todd coupling |
| c_9.7.273 | Oxo to selenoxo | c_2.1.20 | Alkene Ritter reaction |
| | Freund-Gustavson cyclopropane | c_9.7.292 | Atherton-Todd reaction |
| c_3.11.79 | synthesis | c_9.7.271 | Aldehyde Schmidt reaction |
| c_9.7.279 | Formyl Tsuji-Wilkinson decarbonylation | c_3.11.30 | Wallach degradation |
| c_2.5.3 | Thioimidic acid + amine reaction | c_3.9.30 | Chloro formaldehyde Barbier reaction |
| c_9.7.301 | Cyano to aminoamidino | c_9.7.62 | Fluoro Gabriel synthesis |
| c_10.4.13 | Woodward cis-hydroxylation | c_3.4.1 | Stille reaction |
| c_3.9.19 | Eglinton reaction | c_3.5.5 | Fukuyama coupling |
| c_3.9.8 | Bromo Nozaki-Hiyama-Kishi reaction | c_3.11.49 | Koch reaction |
| c_3.9.71 | Epoxide Corey-Seebach reaction | c_4.3.13 | Thiazolidin-4-one synthesis |
| c_9.7.235 | Dithiane to difluoro | c_3.9.20 | Glaser coupling |
| c_9.7.76 | Iodo Gabriel synthesis | c_9.7.144 | Fluoro to formyl |
| c_1.6.5 | Fluoro Gabriel alkylation | c_9.7.97 | Sandmeyer chlorination |
| c_3.11.40 | Stobbe condensation | c_9.7.272 | Riessert hydrolysis |
| c_3.9.7 | Crabbe homologation | c_3.9.58 | Buchner ring expansion |
| c_6.5.5 | Aldehyde dithiolane deprotection | c_3.9.49 | Chloro Takai olefination |
| c_4.2.38 | Simonis chromone cyclization | c_9.7.203 | Iodo Hunsdiecker reaction |
| c_3.11.28 | Bergmann amino acid synthesis | c_9.7.274 | Diazonio to azido |
| c_3.9.10 | Iodo Nozaki-Hiyama-Kishi reaction | c_4.1.81 | Reductive ring contraction |
| c_9.7.281 | Chlorocarbonyl decarbonylation | c_4.1.88 | Orru imidazoline synthesis |
| c_9.7.121 | Diazonio to fluoro | c_9.7.189 | Lossen rearrangement |
| c_10.1.14 | Hell-Volhard-Zelinsky halogenation | c_9.7.296 | Acid chloride to peroxyacid |
| c_4.2.46 | Achmatowicz reaction | c_9.7.108 | Chlorosulfonyl to chloro |
| c_9.7.50 | Chlorosulfinyl to sulfinamoyl | c_10.1.16 | Zincke sulfur chlorination |
| c_4.1.38 | Gewald pyrrole synthesis | c_9.7.243 | Iodoform reaction |
| c_11.8.6 | Lithium salt formation | c_3.11.9 | Seyferth-Gilbert ketone homologation |
| c_3.9.33 | Birch alkylation | c_6.3.14 | Mann ether demethylation |
| c_8.1.17 | Oppenauer-Woodward oxidation | c_9.7.131 | Fluorosulfonyl to sulfo |
| c_8.1.22 | Fluoro to oxo oxidation | c_8.8.13 | Boyland-Sims oxidation |
| c_3.9.32 | Acyloin condensation | c_10.3.3 | Alkene sulfoxy addition |
| | Johnson-Corey-Chaykovsky aziridine | c_9.7.308 | Zinner hydroxylamine synthesis |
| c_4.1.33 | synthesis | c_4.1.25 | Camps quinoline synthesis |
| c_4.1.43 | Indazolone synthesis | c_3.9.15 | Weinreb chloro coupling |
| c_3.3.6 | Fluoro Sonogashira coupling | c_3.5.6 | Hiyama-Denmark coupling |
| c_7.9.7 | Diazonio to hydrazino reduction | c_9.7.199 | Iodo Kochi reaction |
| c_8.8.12 | Elbs persulfate oxidation | | Carboxylic anhydride + sulfinamide |
| c_4.1.41 | Baeyer-Emmerling indole synthesis | c_2.1.13 | reaction |
| c_4.1.51 | Kroehnke pyridine synthesis | c_9.7.282 | Cyanocarbonyl decarbonylation |
| c_8.1.19 | Aldehyde Corey-Kim oxidation | c_9.5.3 | Chugaev elimination |
| c_3.9.57 | Gatterman-Koch formylation | c_10.1.18 | Zincke sulfur bromination |
| c_9.7.294 | Acid to peroxyacid | c_3.11.61 | Fluoro Nierenstein reaction |
| c_9.7.280 | Bromocarbonyl decarbonylation | c_9.7.119 | Diazonio to bromo |
| c_11.9.2 | Potassium salt separation | c_8.8.15 | Aldehyde autoxidation |
| c_9.7.156 | Bromo Kolbe isonitrile synthesis | c_3.9.39 | Aldehyde Nef synthesis |
| | | c_1.2.7 | Aldehyde Leuckart reaction |

1. Chemical reaction encoding software for one-step, multi-step and equilibrium reactions, characterised in that it executes instructions corresponding to the following steps:
a step (105) of receiving, upon a computer interface, a chemical reaction graph comprising at least one chemical reaction reagent and at least one chemical reaction product,
a first step (110) of encoding, by a computing device, said chemical reaction graph describing the structure of at least one said reagent and said product,
a step (115) of determination, by a computing device, of changing bonds within the encoding representative of the chemical structures of said at least one reaction reagent and said product,
a second step (120) of encoding, by a computing device, in a single string of characters, for at least one changing bond determined, at least one character representative of an atom subject to the change of bond, at least one character representative of the type of changing bond determined and at least one character representative of an atom resulting from the change of bond, in which a changing bond is encoded by a set of two characters representative of the changing bond determined, the first character being representative of the reagent bond and the second character being representative of the product bond, each character being selected in a library of bijective characters wherein one character is representative of one bond type and
a step (125) of providing, upon a computer interface, the string of characters corresponding to the encoding of changing bonds of the chemical reaction.

2. Software according to claim 1, in which the second (120) step of encoding is configured to embed the two characters representative of the changing bonds determined in between two neutral tag characters representative of the presence of an encoding of said changing bonds.

3. Software according to claim 1, in which multistep reactions, represented by a succession of change of bonds between two atoms, are encoded by a succession of single characters, each single character being representative of the successive state of a bond between said two atoms, the order of the characters being representative of the order of changes of bonds between said two atoms.

4. Chemical reaction encoding method (100) for one-step, multi-step and equilibrium reactions, characterised in that it comprises:
a step (105) of receiving, upon a computer interface, a chemical reaction graph comprising at least one chemical reaction reagent and at least one chemical reaction product,
a first step (110) of encoding, by a computing device, said chemical reaction graph describing the structure of at least one said reagent and said product,
a step (115) of determination, by a computing device, of changing bonds within the encoding representative of the chemical structures of at least one said reaction reagent and said product,
a second step (120) of encoding, by a computing device, in a single string of characters, for at least one changing bond determined, at least one character representative of an atom subject to the change of bond, at least one character representative of the type of changing bond determined and at least one character representative of an atom resulting from the change of bond, in which a changing bond is encoded by a set of two characters representative of the changing bond determined, the first character being representative of the reagent bond and the second character being representative of the product bond, each character being selected in a library of bijective characters wherein one character is representative of one changing bond type and
a step (125) of providing, upon a computer interface, the string of characters corresponding to the encoding of changing bonds of the chemical reaction.

5. Method (100) according to claim 4, in which multistep reactions, represented by a succession of change of bonds between two atoms, are encoded by a succession of single characters, each single character being representative of the successive state of a bond between said two atoms, the order of the characters being representative of the order of changes of bonds between said two atoms.

6. Method (100) according to claim 4, in which the first step (110) of encoding is configured to encode the chemical reaction graph into a line notation, the method further comprising, prior to the second step (120) of encoding, a step (130) of augmenting the line notation encoding.

7. Method (100) according to claim 4, in which the second step (120) of encoding comprises a step (121) of extracting, by a computing device, of a bond table for reagents and products from a computer memory, said encoding being performed as a function of said bond table.

8. Method (100) according to claim 4, in which the second step (120) of encoding comprises a step of removing, from the first encoding resulting from the first step (110) of encoding, of at least one atom identifier from at least one reagent and/or product, each said atom being removed as a result of the step (115) of determination in the event said atom and the associated bonds are located in a product and/or reagent that remains unchanged from reagent the reaction stage to the product stage of the chemical reaction.

9. Method (100) according to claim 4, which comprises a step (135) of obtaining the products of the encoded chemical reaction by performing said chemical reaction in a physical device.

10. Encoded chemical reaction comprising a string of characters (205, 210), characterised in that it is obtained by the method (100) according to claim 4.

11. Chemical reaction dataset augmentation method (300), characterised in that it comprises:
a step (305) of receiving, upon a computer interface, a string of characters according to the encoding of claim 10,
a step (310) of reordering, by a computing system, the string of characters in order to shift at least one character representative of an atom and at least one string of at least one character representative of a change of bond associated to the corresponding atom and
a step (320) of outputting, upon a computer interface, an augmented string of characters corresponding to the reaction initially encoded by the received string of characters.

12. Augmentation method (300) according to claim 11, which further comprises a step (315) of associating, by a computing system, at least two string of characters according to the format of claim 9, each said string of characters being representative of the same chemical reaction graph.

13. Chemical reaction dataset preprocessing method (400), characterised in that it comprises:
a step (405) of receiving, upon a computer interface, a dataset of at least two chemical reaction graphs comprising at least one chemical reaction reagent and at least one chemical reaction product,
a step (100) of compression of at least two chemical reaction graphs according to the method according to claim 4,
a step (410) of determining, by a computing system, a distribution of chemical reaction classes within the encoded dataset,
a step (300) of augmenting the dataset, wherein the augmenting comprises either (A) a step (121) of extracting, by a computing device, of a bond table for reagents and products from a computer memory, said encoding being performed as a function of said bond table or (B) a step of removing, from the first encoding resulting from the first step (110) of encoding, of at least one atom identifier from at least one reagent and/or product, each said atom being removed as a result of the step (115) of determination in the event said atom and the associated bonds are located in a product and/or reagent that remains unchanged from reagent the reaction stage to the product stage of the chemical reaction, for at least one chemical reaction class as a function of the determined distribution and
a step (415) of outputting, upon a computer interface, the preprocessed dataset.

14. Training method (500) for a classifier, transformer or regressor, characterised in that it comprises:
a step (505) of inputting, upon a computer interface, a dataset of chemical reaction graphs encoded in the compressed encoding according to claim 8,
a step (510) of operating, by a computing system, a recursive neural network architecture configured to use, as input, the dataset of chemical reaction graphs to classify the chemical reaction bond evolution as a function of the input and
a step (515) of outputting, upon a computer interface, a trained classifier, transformer or regressor.

15. Chemical reaction bond evolution prediction method, characterised in that it operates a classifier, transformer or regressor obtained by the method (500) according to claim 14.

16. Chemical reaction generation method, characterised in that it operates a classifier, transformer or regressor obtained by the method (500) according to claim 14.

17. Computer implemented classifier, characterised in that the classifier, transformer or regressor is obtained by the method (500) according to claim 14.

18. Computer program, characterised in that it comprises instructions to operate a method (500) according to claim 14.
